(19) United States
(12) Patent Application Publication
(10) Pub. No.: US 2018/0010117 A1
(43) Pub. Date: Jan. 11, 2018

(54) TARGETED TREATMENT OF LEBER CONGENITAL AMOUROSIS

(71) Applicant: Sangamo Therapeutics, Inc., Richmond, CA (US)

(72) Inventor: David Paschon, Richmond, CA (US)

(21) Appl. No.: 15/642,165

(22) Filed: Jul. 5, 2017

Related U.S. Application Data
(60) Provisional application No. 62/359,433, filed on Jul. 7, 2016.

(51) Int. Cl. C12N 15/10; A61K 38/46; C12N 15/11; C12N 5/09; C12N 9/22; A61K 9/00

(52) U.S. Cl. CPC C12N 15/102; C12Y 301/00; C12N 9/22; A61K 9/0048; C12N 15/11; C12N 5/0694; A61K 38/465; C12N 2310/20; C12N 2510/04

(57) ABSTRACT

Disclosed herein are methods and compositions for inactivating mutant genes associated with LCA, using engineered nucleases comprising a DNA binding domain and a cleavage domain or cleavage half-domain in conditions promoting the cleavage of the mutant genes. Polynucleotides encoding nucleases, vectors comprising polynucleotides encoding nucleases, and cells comprising polynucleotides encoding nucleases and/or cells comprising nucleases are also provided.

### CROSS-REFERENCE TO RELATED APPLICATIONS

The present application claims the benefit of U.S. Provisional Application No. 62/359,433, filed Jul. 7, 2016, the disclosure of which is hereby incorporated by reference in its entirety.

### TECHNICAL FIELD

The present disclosure is in the field of genome modification of human cells, including retinal cells and structures found in the eye.

### BACKGROUND

Gene therapy holds enormous potential for a new era of human therapeutics. These methodologies will allow treatment for conditions that have not been addressable by standard medical practice. Gene therapy can include the many variations of genome editing techniques such as disruption or correction of a gene locus, and insertion of an expressible transgene that can be controlled either by a specific exogenous promoter fused to the transgene, or by the endogenous promoter found at the site of insertion into the genome.

Recombinant transcription factors comprising the DNA binding domains from zinc finger proteins (“ZFPs”), TAL-effector domains (“TALEs”) and CRISPR/Cas transcription factor systems have the ability to regulate gene expression of endogenous genes (see, e.g., U.S. Pat. Nos. 8,586,526; 6,534,261; 6,599,692; 6,503,717; 6,689,558; 7,067,317; 7,262,054; Perez-Pinera et al. (2013) Nature Methods 10:973-976; Platek et al. (2014) Plant Biotechnology J. doi: 10.1111/pbi.12284). Clinical trials using these engineered transcription factors containing zinc finger proteins have shown that these novel transcription factors are capable of treating various conditions. (see, e.g., Yu et al. (2006) FASEB J. 20:479-481).

In addition, artificial (engineered) nucleases comprising the DNA binding domains from zinc finger proteins (“ZFPs”), TAL-effector domains (“TALEs”), Ttago and CRISPR/Cas nuclease systems (including Cas and/or Cfp1) have the ability to modify gene expression of endogenous genes via nuclease-mediated modification of the gene. In some instances, cleavage by the artificial nuclease causes a double strand break in the chromosome that can be repaired by the error-borne NHEJ process. NHEJ can introduce small insertions and/or deletions (“indels”) at the cleavage site in the gene, and thus cause a knock-out of gene expression and function. In addition, following cleavage driven by an artificial nuclease, an exogenous sequence can be introduced into the cleavage site where incorporation of the exogenous sequence occurs through either homology directed repair (HDR), following non-homologous end joining (NHEJ) and/or by end capture during non-homologous end joining (NHEJ) driven processes. See, e.g., U.S. Pat. Nos. 9,394,531; 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publication Nos. 2003/0232410; 2005/0208489; 2005/0026157; 2005/0064474; 2006/0063231; 2008/0159996; 2010/00218264; 2012/0017290; 2011/0265198; 2013/0137104; 2013/0122591; 2013/0177983; 2013/0196373; 2015/0056705; 2015/0335708; 2016/0030477; and 2016/0024474. Further, targeted nucleases are being developed based on the Argonaute system (e.g., from T. thermophilus, known as ‘TtAgo’, see Swarts et al (2014) Nature 507(7491): 258-261), which also may have the potential for uses in genome editing and gene therapy. This nuclease-mediated approach to transgene integration offers the prospect of improved transgene expression, increased safety and expressional durability, as compared to classic integration approaches, since it allows exact transgene positioning for a minimal risk of gene silencing or activation of nearby oncogenes.

Leber congenital amaurosis (LCA) is a name given to a group of retinal diseases characterized by poor vision in the first year following birth and a number of other vision problems such as photophobia (sensitivity to light), nystagmus (involuntary movements of the eye) and hyperopia (extreme farsightedness) for which there is currently no cure (Burnight et al (2014) Gene Ther 21(7):662-672). LCA occurs in 2 or 3 children for every 100,000 newborns and is one of the most common causes of blindness in children (approximately 20%). Thus far, mutants in at least fourteen different human genes including aryl hydrocarbon receptor interacting protein like 1 (AIPL1); Centrosomal protein 290 (CEP290); Crumbs1 cell polarity complex component (CRB1); Cone-rod homeobox (CRX); Guanylate cyclase 2D (GUCY2D); Inosine monophosphate dehydrogenase 1 (IMPDH1); Lebercilin (LCAS); Lecithin retinol acyltransferase (LRAT); Retinal degeneration 3 (RD3); Retinol dehydrogenase 12 (RDH12); Retinal pigment epithelium specific protein 65 (RPE65); Retinitis pigmentosa GTPase regulator interacting protein 1 (RPGRIP1); Spermatogenesis associated 7 (SPATA7); and Tubby like protein 1 (TULP1), account for 70% of patients with European ancestry (Coppieters et al (2010) Hum Mut, 31(10) E1709-E1766). Treatment of LCA arising from mutations in RPE65 has been explored clinically where subretinal delivery of an AAV carrying a RPE65 cDNA resulted in modest improvements in retinal function (Maguire et al (2008) N Engl J Med 358(21): 2240-2248). Follow up studies of up to 3 years post RPE65 therapy have continued to show safety of the treatment and modest efficacy (Jacobson et al (2015) Exp Opin Orph Drugs 3(5):563-575).

Approximately 30% of LCA patients harbor mutations in CEP290, making CEP290 mutations the largest contributor to the development of LCA (Burnight, ibid). CEP290 is considered a disease gene that contributes to a set of disorders known as ciliopathies. Cilia are highly conserved organelles with well-known roles in motility and transport of fluids and particles over epithelial surfaces, yet they have roles in other processes such as signal transduction as well (Coppieters et al (2010) Hum Mut 31(10): 1097-1108). Since cilia are present throughout the whole body, it is not surprising that ciliary proteins are thought to play roles in a variety of different diseases. For example, mutations in CEP290 are thought to be involved in diseases with renal, kidney, neural tube, central nervous system and/or bone involvement, such as lethal Meckel-Gruber syndrome (MKS), Joubert syndrome, Senior-Loken syndrome, LCA, and Bardet-Biedl syndrome (Coppieters, ibid, and Gerard et al (2015) Mol Ther Nuc Acid 4, e250)). Thus far, over 112 unique CEP290 mutations have been identified in humans. The majority of these mutations are truncating mutations, caused by either missense or nonsense mutations.

One CEP290 mutation that appears to be a causal mutation for LCA is the c.2991+1655A>G mutation, particularly in patients with northern European ancestry. Interestingly, this mutation does not appear to be highly prevalent in LCA patients with Italian, Saudi Arabian, Spanish, Indian or Korean roots. In addition, this particular mutation may play only a minor, if any, role in other diseases related to CEP290. The mutation itself, a substitution of an adenine for a guanine at position 1655 within intron 26, activates a cryptic splice donor site downstream of a normally present strong acceptor splice site. This new splice donor site results in the insertion of a 128 bp intronic sequence within the CEP290 transcript, where the inserted sequence comprises a stop codon (Gerard et al, ibid). Loss of CEP290 appears to lead to a rapid reduction in photoreceptor outer segment length and outer nuclear layer thickness in the eye. However, LCA CEP290 patients retain substantial central cone photoreceptors (McAnany et al (2013) JAMA Ophthalmol 131(2): 178-182).

Thus, there remains a need for methods and compositions that can be used to prevent and/or treat LCA through treatments that rely on specific gene targeting to alter gene expression.

### SUMMARY

Disclosed herein are compositions (e.g., artificial nucleases and transcription factors) and methods for partial or complete restoration of normal splicing of a gene whose mis-splicing (and consequent mis-translation) is involved in LCA. In certain embodiments, the compositions are delivered to cells such that a key, aberrant splicing signal is inactivated (e.g., knocked out) and, following inactivation, the LCA is treated in the subject (via ex vivo or in vivo methods).

Thus, in one aspect, described herein are cells in which the expression of a gene involved in LCA is modulated. In some embodiments, modified cells are described that comprise an engineered nuclease to cause a knockout of a gene or critical gene element (e.g. a splicing signal) such that the LCA is treated. Cells which are modified (e.g., genetic modifications to the sequence of the LCA gene) by such nucleases (e.g., genetic modifications within the nuclease target(s)) are also provided for the treatment of LCA. In other embodiments, cells are described that comprise an engineered transcription factor (TF) such that the expression of a gene related to LCA is modulated. In preferred embodiments, the gene to be targeted by the nuclease(s) and/or transcription factor(s) is the aryl hydrocarbon receptor interacting protein like 1 (AIPL1); Centrosomal protein 290 (CEP290); Crumbs1 cell polarity complex component (CRB1); Cone-rod homeobox (CRX); Guanylate cyclase 2D (GUCY2D); Inosine monophosphate dehydrogenase 1 (IMPDH1); Lebercilin (LCAS); Lecithin retinol acyltransferase (LRAT); Retinal degeneration 3 (RD3); Retinol dehydrogenase 12 (RDH12); Retinal pigment epithelium specific protein 65 (RPE65); Retinitis pigmentosa GTPase regulator interacting protein 1 (RPGRIP1); Spermatogenesis associated 7 (SPATA7); or Tubby like protein 1 (TULP1). In preferred embodiments, the gene to be modified comprises a mutation that is linked to LCA. In some instances, the LCA associate gene mutation is in the CEP290 gene. In especially preferred embodiments, the CEP290 mutation is the c.2991+1655A>G mutation. Particularly preferred is the use of an engineered nuclease to cleave at or near a CEP290 mutation (e.g. the c.2991+1655A>G mutation) such that an aberrant splicing signal is destroyed during NHEJ which follows nuclease driven cleavage, restoring normal splicing to the CEP290 transcript. Alternatively, engineered nucleases can be used to cleave at or near the CEP290 mutation and a donor molecule with the wild-type sequence can be inserted (integrated) into the gene to restore normal splicing of the CEP290 transcript.

In one aspect, described herein a modified cell in which expression of an aberrant endogenous Leber congenital amaurosis (LCA)-related gene is partially or completely restored as compared to a cell comprising an aberrant endogenous LCA-related gene that is not modified. In certain embodiments, the LCA-related gene is an aryl hydrocarbon receptor interacting protein like 1 (AIPL1) gene; a Centrosomal protein 290 (CEP290) gene; a Crumbs1 cell polarity complex component (CRB1) gene; a Cone-rod homeobox (CRX) gene; a Guanylate cyclase 2D (GUCY2D) gene; an Inosine monophosphate dehydrogenase 1 (IMPDH1) gene; a Lebercilin (LCAS) gene; a Lecithin retinol acyltransferase (LRAT) gene; a Retinal degeneration 3 (RD3) gene; a Retinol dehydrogenase 12 (RDH12) gene; a Retinal pigment epithelium specific protein 65 (RPE65) gene; a Retinitis pigmentosa GTPase regulator interacting protein 1 (RPGRIP1) gene; a Spermatogenesis associated 7 (SPATA7) gene; or a Tubby like protein 1 (TULP1) gene. Gene expression in the cell may be modulated by genetic modification of the gene (e.g., sequence modification by a nuclease such as a ZFN, a TALEN, a CRISPR/Cas nuclease system, etc.), for example by insertions and/or deletions. Alternatively, or in addition to, genetic modification, gene expression may be modified by binding to the gene (e.g., an artificial transcription factor that increases or decreases gene expression such as a ZFP-TF, a TALE-TF, A CRISPR/Cas-TF, etc.). In certain embodiments, a CEP290 gene is genetically modified by introducing an insertion and/or deletion into the CEP290 gene, for example, an insertion and/or deletion is within a target site as shown in Table 1 or within 300 nucleotides of a target site as shown in Table 1. In certain embodiments, the artificial transcription factor comprises a transcriptional regulatory domain (e.g., a repression domain or an activation domain) and a DNA-binding domain that binds to a target site comprising 12 or more base pairs as shown in Table 1.

In certain embodiments, the modified cells described herein comprise a modification (e.g., deletion, insertion and/or correction of a point mutation) to a gene involved in LCA in which the modification is within, at or near nuclease(s) binding and/or cleavage site(s), including but not limited to, modifications to sequences within 1-300 (or any number of base pairs therebetween) base pairs upstream, downstream and/or including 1 or more base pairs of the site(s) of cleavage and/or binding site; modifications within 1-100 base pairs (or any number of base pairs therebetween) of including and/or on either side of the binding and/or cleavage site(s); modifications within 1 to 50 base pairs (or any number of base pairs therebetween) including and/or on either side of the binding and/or cleavage site(s); and/or modifications to one or more base pairs within the nuclease binding site and/or cleavage site, including within or between paired target (binding) sites such as those shown in Table 1 (exemplary pairs shown in Table 2). In other embodiments, described herein are modified cells comprising an artificial transcription factor (e.g., an artificial transcription factor comprising a DNA-binding domain and a transcriptional regulatory domain) that alters (increases or decreases) expression of an LCA-related gene, for example by binding to the LCA-related gene to form a complex with the target gene, thereby altering gene expression of the LCA-related gene.

The modified cells of the invention may be a stem/progenitor cell (e.g., an induced pluripotent stem cell (iPSC), an embryonic stem cell (e.g., human ES), a mesenchymal stem cell (MSC), a hematopoietic stem cell (HSC), or a mesenchymal stem cell). The stem cells may be totipotent or pluripotent (e.g., partially differentiated such as an HSC that is a pluripotent myeloid or lymphoid stem cell or a mesenchymal stem cell that differentiates into a epithelial stem cell and/or stem cell). Any of the modified stem cells described herein (modified at a locus related to LCA) may then be differentiated to generate a differentiated (in vivo or in vitro) cell descended from a stem cell as described herein. Any of the modified stem cells described herein may be comprise further modifications in other genes of interest).

Thus, described herein is a composition for modifying one or more aberrant endogenous LCA-related genes in a cell of a mammalian subject (e.g., a subject suffering from LCA), the composition comprising one or more nucleases, the one or more nuclease(s) comprising a DNA-binding domain that binds to the aberrant endogenous LCA-related gene and an endonuclease cleavage domain, wherein the nuclease cleaves the one or more endogenous LCA-related gene. In certain embodiments, the modification is selected from the group consisting of an insertion, a deletion, a substitution and combinations thereof. Any of the compositions described herein can result one or more aberrant endogenous LCA-related genes is partially or completely restored, for example by modification of an aberrant endogenous LCA-related gene (e.g., CEP290) in one or more cells of the subject.

In another aspect, the compositions (modified cells, polynucleotides and/or proteins used to modulate LCA-related gene expression) and methods described herein can be used, for example, in the treatment, prevention and/or amelioration of a disorder such as LCA. The methods typically can comprise any modification (up-regulation, down-regulation, cleaving, etc.) of an endogenous gene associated with LCA in an isolated cell or tissue of a subject using an engineered transcription factor and/or nuclease (e.g., ZFN or TALEN) or nuclease system such as CRISPR/Cas or Cfp1/CRISPR with an engineered crRNA/tracr RNA, or using an engineered transcription factor (e.g. ZFN-TF, TALE-TF, Cfp1-TF or Cas9-TF) such that the gene is modulated (up-regulated, down-regulated, inactivated); and (b) introducing the cell into the subject, or introducing the polynucleotides and/or proteins used to modulate LCA-related genes into the eye, thereby treating or preventing LCA. Thus, described herein is a method of treating or preventing LCA in a subject, the method comprising: administering a nuclease that modifies an LCA-related gene to generate a genetically modified cell according to claim 3, wherein the cell is in the subject and LCA is treated or prevented. The genetically modified cell may be generated in vitro and administered to the subject (e.g., ex vivo methods). Administration of the cell may be achieved by any means, including via topical application and/or via injection into the area to be treated. Also described herein is a method of treating LCA in a subject comprising administering a composition as described herein to the subject, for example via administration to the eye. In certain embodiments, the method comprises generating a cell in the subject by administering to the subject an artificial transcription factor that alters expression of an LCA-related gene.

In any of the compositions and methods described herein, the nuclease or transcription factor may comprise one or more zinc finger proteins (ZFP-TFs or ZFNs), one or more TAL-effector domain nucleases (TALE-TFs or TALENs), and/or one or more components of a TtAgo or CRISPR/Cas transcription factor or nuclease system (e.g. the Cas protein and/or the sgRNA). In addition, the compositions and methods described herein may be made or practiced in vivo or ex vivo, including, but not limited to, mammalian cells such as K562 cells, Hepa1-6 cells, CD4+ T cells, CD8+ T cells, CD34+ hematopoietic stem cells (HPSCs), and in vivo (e.g., retinal cells); yeast cells such as S. cerevisiae or S. pichia; insect cells such as SF-9 cells, and plant cells derived from maize, wheat or canola. Transcription factors and/or nucleases as described herein are designed to recognize (and/or cleave) a sequence at or near the LCA mutation in intron 26, for example within, at or near a site comprising 12 or more (e.g., 12-35 or any value therebetween) contiguous nucleotides of any of the sequences shown in FIG. 1 (SEQ ID NO:35), FIG. 2 (SEQ ID NO:36-73), FIG. 3 (SEQ ID NO:74-111) and/or the nuclease target sites of Table 1 (SEQ ID NO:1-5).

Thus, described herein is a DNA-binding domain (e.g., zinc-finger protein (ZFP), TALE effector domain, or single guide RNA of a CRISPR/Cas system) that binds to target site in a LCA-related gene in a genome. In certain embodiments, the target site recognized by the DNA-binding domain is in CEP290 gene. The target site may be in an intron or exon of the targeted gene. In certain embodiments, the target site comprises a sequence of 12-25 (including target sites of 12, 13, 14, 15, 16, 17, 18, 19, 20, 21, 22, 23, 24, 25) or more nucleotides as shown in the target sites of Table 1 or a target sequence in the same gene from a related species, including target sites from other species (e.g., human) that exhibit 80%, 85%, 90%, 95%, 96%, 97%, 98% or 99% or more homology or hybridize under stringent conditions (as known in the art) to the target sites shown in Table 1. In certain embodiments, the target site is in a human gene. In certain embodiments, the DNA-binding domain comprises a zinc finger protein with the recognition helix regions as shown in a single row of Table 1 (recognition helix regions of F1-F5 or F1-F6). In other embodiments, the DNA-binding domain comprises a TALE or an sgRNA.

In one embodiment, the DNA-binding domain is in association (e.g., as a fusion protein, or interacts with, in the case of a single guide RNA) with a functional domain to form an artificial transcription factor (e.g., where the functional domain is a transcriptional regulatory domain or is an inactive (e.g. ‘dead’) nuclease domain) or an artificial nuclease (e.g., where the functional domain is a cleavage domain). The transcriptional regulatory domain may be an activation domain or a repression domain.

In other embodiments, the DNA-binding domain is in association with at least one cleavage domain (or cleavage half-domain) to form an artificial nuclease. Cleavage domains and cleavage half domains can be obtained, for example, from various restriction endonucleases and/or homing endonucleases. In one embodiment, the cleavage half-domains are derived from an endonuclease, for example a Type IIS restriction endonuclease (e.g., Fok I) and/or a Cas endonuclease. In certain embodiments, the DNA-binding domain recognizes a target site in an LCA-related (e.g., CEP290) gene (e.g., a target site comprising 12 or more nucleotides of a target site as shown in Table 1, including for example, a zinc finger protein comprising the recognition helix regions as shown in a single row of Table 1 or a TALE or sgRNA that binds to a target sequence encompassed in a target site of Table 1). In further embodiments, the DNA binding domain recognizes a target site in an LCA-related gene where the LCA-related gene is a human gene.

The DNA-binding domains, artificial TFs and/or artificial nucleases may bind to and/or cleave the target gene within the coding region of the gene or in a non-coding sequence within or adjacent to the gene, such as, for example, a leader sequence, trailer sequence or intron, or within a non-transcribed region, either upstream or downstream of the coding region. In certain embodiments, the DNA-binding domains as described herein bind to sequence within an intron of the target gene (e.g., intron 26 of CEP290). In other embodiments, the DNA-binding domains as described herein bind to sequence within an exon of the target gene. Any DNA-binding domain, artificial TF and/or artificial nuclease may be used provided such DNA binding domain, TF and/or nuclease binds to or cleaves the target gene. In some embodiments, the methods and compositions of the invention involve the use of a DNA bind domain, artificial TF and/or artificial nuclease for the treatment of LCA in a patient in need thereof.

In yet another aspect, a polynucleotide encoding one or more of the DNA binding proteins or fusion molecules (or components thereof) described herein is provided. In certain embodiments, the polynucleotide is carried on a viral (e.g., AAV or Ad) vector and/or a non-viral (e.g., plasmid or mRNA vector). Host cells comprising these polynucleotides (e.g., AAV vectors) and/or pharmaceutical compositions comprising the polynucleotides, proteins and/or host cells as described herein are also provided. Host cells include but are not limited to T-cell and stem cells such as skin stem cells.

In some embodiments, the polynucleotide encoding the DNA binding protein is an mRNA. In some aspects, the mRNA may be chemically modified (See e.g. Kormann et al, (2011) Nature Biotechnology 29(2):154-157). In other aspects, the mRNA may comprise an ARCA cap (see U.S. Pat. Nos. 7,074,596 and 8,153,773). In further embodiments, the mRNA may comprise a mixture of unmodified and modified nucleotides (see U.S. Patent Publication No. 2012/0195936).

In another aspect, the invention provides kits that are useful for treating LCA comprising engineered transcription factors and/or nucleases (e.g., ZFP-TFs, TALE-TFs, ZFNs, TAL-effector domain nuclease fusion proteins, engineered homing endonucleases, Ttago, CRISPR/Cas transcription factor, sgRNA or nuclease systems). The kits typically include one or more nucleases that bind to a target site in a gene associated with LCA and introduce a specific double strand break.

The nuclease(s) and/or transcription factor(s) can be introduced as mRNA, in protein form and/or as a DNA sequence encoding the nuclease(s), including non-viral and viral vectors (e.g., AAV). In some embodiments, the nuclease(s) or transcription factor(s) are introduced into the eye as mRNAs, while in others, they are introduced as DNA. In some embodiments, delivery is accomplished via subretinal injection of a nucleic acid (e.g., viral vector), (see for example only, You et al (2014) PLOS ONE 9(8) e104145), a nanoparticle (for example only see McCaffrey et al (2016) J Contr Release 226: 238) or liposome (see Desmet et al (2016) Int J Pharm. 500(1-2):268-74). In certain embodiments, isolated stem cells comprising the nuclease or transcription factor are introduced into the subject, while in others, the isolated stem cells are treated first ex vivo and then introduced into the subject (see Mistriotis and Andreadis (2013) Tiss Engineer 19(4):265).

Any of the proteins described herein may further comprise a functional domain, such as a transcriptional regulatory domain (activation domain, repression domain) or a nuclease domain (cleavage domain and/or a cleavage half-domain (e.g., a wild-type or engineered FokI cleavage half-domain)). Thus, in any of the transcription factors and/or nucleases described herein, the nuclease domain may comprise a wild-type functional domain or an engineered functional domain (e.g., engineered FokI cleavage half domains that form obligate heterodimers). See, e.g., U.S. Patent Publication No. 2008/0131962.

In another aspect, the disclosure provides a polynucleotide encoding any of the proteins described herein. Any of the polynucleotides described herein may also comprise sequences (donor or patch sequences) for targeted insertion into a gene (e.g., LCA-gene, for instance to correct a point mutation and/or a wild-type gene into a safe harbor gene). In yet another aspect, a gene delivery vector comprising any of the polynucleotides described herein is provided. In certain embodiments, the vector is an adenoviral vector (e.g., an Ad5/F35 vector) or a lentiviral vector (LV) including integration competent or integration-defective lentiviral vectors or an adeno-associated vector (AAV). Thus, also provided herein are viral vectors comprising a sequence encoding a transcription factor and/or nuclease (e.g. ZFN or TALEN and/or a nuclease system (CRISPR/Cas or Ttago) and/or a donor sequence for targeted integration into a target gene. In some embodiments, the donor sequence and the sequences encoding the nuclease are on different vectors. In other embodiments, the nucleases are supplied as polypeptides. In preferred embodiments, the polynucleotides are mRNAs. In some aspects, the mRNA may be chemically modified (See e.g. Kormann et al, (2011) Nature Biotechnology 29(2):154-157). In other aspects, the mRNA may comprise an ARCA cap (see U.S. Pat. Nos. 7,074,596 and 8,153,773). In further embodiments, the mRNA may comprise a mixture of unmodified and modified nucleotides (see U.S. Patent Publication No. 2012/0195936).

In yet another aspect, the disclosure provides an isolated cell comprising any of the proteins, polynucleotides and/or vectors described herein. In certain embodiments, the cell is a stem/progenitor cell. In a still further aspect, the disclosure provides a cell or cell line which is descended from a cell or line comprising any of the proteins, polynucleotides and/or vectors described herein, namely a cell or cell line descended (e.g., in culture) from a cell in which a gene involved in LCA has been modulated by a engineered transcription factor and/or engineered nuclease (e.g., in which a donor polynucleotide has been stably integrated into the genome of the cell). Thus, descendants of cells as described herein may not themselves comprise the proteins, polynucleotides and/or vectors described herein, but, in these cells, at least one gene involved in LCA is modulated. In some embodiments, the methods and compositions of the invention involve the use of an isolated cell comprising a DNA bind domain, artificial TF and/or artificial nuclease for the treatment of LCA in a patient in need thereof.

In another aspect, described herein are methods of modulating (inactivating, correcting a point mutation, down regulating or up regulating) a gene related to LCA in a cell by introducing one or more proteins, polynucleotides and/or vectors into the cell as described herein, optionally in the presence of a donor. In any of the methods described herein the engineered transcription factor may up or down-regulate expression of one or more genes associated with retinal function and the nucleases may induce targeted mutagenesis, deletions of cellular DNA sequences, and/or facilitate targeted recombination at a predetermined chromosomal locus. Thus, in certain embodiments, the nucleases delete or insert one or more nucleotides of the target gene. In some embodiments the gene is inactivated by nuclease cleavage followed by non-homologous end joining. In other embodiments, a genomic sequence in the target gene is replaced, for example using a nuclease (or vector encoding said nuclease) as described herein and a “donor” sequence that is inserted into the gene following targeted cleavage with the nuclease, for example to correct a mutation such as a point mutation. The donor sequence may be present in the nuclease vector, present in a separate vector (e.g., AAV, Ad or LV vector) or, alternatively, may be introduced into the cell using a different nucleic acid delivery mechanism.

Furthermore, any of the methods described herein can be practiced in vitro, in vivo and/or ex vivo. In certain embodiments, the methods are practiced ex vivo, for example to modify stem cells, to make the cells useful as therapeutics in an allogenic setting to treat a subject (e.g., a subject with LCA).

### BRIEF DESCRIPTION OF THE DRAWINGS

FIG. 1 (SEQ ID NO:35) depicts the gene sequence of CEP290 within the mutated intron 26 where the mutated base is boxed. The ZFN binding targets for three sites (1-3) are indicated with either dashed, grey, or black lines, respectively. The center of the cleavage site for each target pair is indicated with a downwards arrow in the same style.

FIG. 2 (SEQ ID NO:36-73 from top to bottom) depicts a sequence alignment of DNA isolated from cells treated with nucleases where the alignment centers on the target mutation, and shows the binding sites for the left and right ZFN. The target in this dataset is Target 1. The figure illustrates the deletions detected from the sequence analysis. 85% of the sequences comprised indels at this location following ZFN treatment.

FIG. 3 (SEQ ID NO:74-111 from top to bottom) depicts a sequence alignment of DNA isolated from cells treated with nucleases where the alignment centers on a “GT” dinucleotide which comprises a key conserved splice donor motif, and shows the binding sites for the left and right ZFN. The target in this dataset is Target 2. The figure illustrates the deletions detected from the sequence analysis. 27% of the sequences comprised indels at this location following ZFN treatment.

### DETAILED DESCRIPTION

Disclosed herein are compositions and methods for preventing or treating LCA, including pharmaceutical composition comprising one or more engineered transcription factors and/or nucleases that modulate expression of one or more genes involved in LCA. Cells modified by these transcription factors and/or nucleases can be used as therapeutics, for example, transplants, to alter (e.g., restore) vision by treatment and/or prevention of an LCA. Additionally, other genes of interest may be inserted into cells in which the gene(s) has been manipulated and/or other genes of interest may be knocked out.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526, incorporated by reference herein in its entirety.

Zinc finger and TALE DNA-binding domains can be “engineered” to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs and binding data. See, for example, U.S. Pat. Nos. 8,586,526; 6,140,081; 6,453,242; and 6,534,261; see also International Publication Nos. WO 98/53058; WO 98/53059; WO 98/53060; WO 02/016536 and WO 03/016496.

A “selected” zinc finger protein or TALE is a protein not found in nature whose production results primarily from an empirical process such as phage display, interaction trap or hybrid selection. See e.g., U.S. Pat. Nos. 5,789,538; 5,925,523; 6,007,988; 6,013,453; 6,200,759; International Publication Nos. WO 95/19431; WO 96/06166; WO 98/53057; WO 98/54311; WO 00/27878; WO 01/60970; WO 01/88197 and WO 02/099084.

“TtAgo” is a prokaryotic Argonaute protein thought to be involved in gene silencing. TtAgo is derived from the bacteria Thermus thermophilus. See, e.g. Swarts et al, ibid, G. Sheng et al., (2013) Proc. Natl. Acad. Sci. U.S.A. 111, 652). A “TtAgo system” is all the components required including e.g. guide DNAs for cleavage by a TtAgo enzyme.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger proteins can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Pat. Nos. 7,888,121; 7,914,796; 8,034,598; 8,623,618 and U.S. Patent Publication No. 2011/0201055, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. They may also include cargo delivery by mechanical forces resulting in cell squeezing in a microfluidic system. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule (e.g., a CRISPR/Cas system in which a single guide RNA associates with a functional domain to modulate gene expression).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

An “aberrant” gene is a gene comprising a mutation (e.g., an insertion, deletion, substitution or the like) such that normal function of the gene and/or gene product is decreased or abolished. Such mutations can cause disruption of the normal coding sequence, interfere with transcription of the gene, cause incorrect splicing of the primary gene transcript and/or cause misfolding of the encoded gene product. These mutations can result in the mis-functioning of the gene and/or gene product.

A “safe harbor” locus is a locus within the genome wherein a gene may be inserted without any deleterious effects on the host cell. Most beneficial is a safe harbor locus in which expression of the inserted gene sequence is not perturbed by any read-through expression from neighboring genes. Non-limiting examples of safe harbor loci that are targeted by nuclease(s) include CCRS, CCRS, HPRT, AAVS1, Rosa and albumin. See, e.g., U.S. Pat. Nos. 7,951,925; 8,771,985; 8,110,379; 7,951,925; U.S. Patent Publication Nos. 2010/0218264; 2011/0265198; 2013/0137104; 2013/0122591; 2013/0177983; 2013/0177960; 2015/0056705 and 2015/0159172.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs. In some embodiments, a region of interest can be up to 3000, 4000, 5000, 7000 or 10000 base pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a DNA-binding domain (e.g., ZFP, TALE) is fused to an activation domain, the DNA-binding domain and the activation domain are in operative linkage if, in the fusion polypeptide, the DNA-binding domain portion is able to bind its target site and/or its binding site, while the activation domain is able to up-regulate gene expression. When a fusion polypeptide in which a DNA-binding domain is fused to a cleavage domain, the DNA-binding domain and the cleavage domain are in operative linkage if, in the fusion polypeptide, the DNA-binding domain portion is able to bind its target site and/or its binding site, while the cleavage domain is able to cleave DNA in the vicinity of the target site. Similarly, with respect to a fusion polypeptide in which a DNA-binding domain is fused to an activation or repression domain, the DNA-binding domain and the activation or repression domain are in operative linkage if, in the fusion polypeptide, the DNA-binding domain portion is able to bind its target site and/or its binding site, while the activation domain is able to upregulate gene expression or the repression domain is able to downregulate gene expression.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and International Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Cancer and graft versus host disease are non-limiting examples of conditions that may be treated using the compositions and methods described herein.

DNA-Binding Domains

Described herein are compositions (e.g., artificial transcription factors and/or nucleases) comprising a DNA-binding domain that specifically binds to a target site in any LCA-related gene, for example a CEP290 gene or a CEP290 regulator. Any DNA-binding domain can be used in the compositions and methods disclosed herein, including but not limited to a zinc finger DNA-binding domain, a TALE DNA binding domain, the DNA-binding portion (sgRNA) of a CRISPR/Cas nuclease, or a DNA-binding domain from a meganuclease. Hybrid DNA binding domains (e.g. megaTALEs, as well as Cas9 fusions with zinc fingers or TALEs) may also be used.

In certain embodiments, the DNA binding domain comprises a zinc finger protein. Preferably, the zinc finger protein is non-naturally occurring in that it is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties. In certain embodiments, the DNA-binding domain comprises a zinc finger protein disclosed in U.S. Patent Publication No. 2012/0060230 (e.g., Table 1), incorporated by reference in its entirety herein.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Publication Nos. WO 98/37186; WO 98/53057; WO 00/27878; WO 01/88197 and GB 2,338,237. In addition, enhancement of binding specificity for zinc finger binding domains has been described, for example, in U.S. Pat. No. 6,794,136.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in U.S. Pat. No. 6,794,136.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; and International Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In certain embodiments, the DNA binding domain is an engineered zinc finger protein that binds (in a sequence-specific manner) to a target site in an CEP290 gene or and knocks out a splicing mutation. In some embodiments, the zinc finger protein binds to a target site in aryl hydrocarbon receptor interacting protein like 1 (AIPL1); Centrosomal protein 290 (CEP290); Crumbs1 cell polarity complex component (CRB1); Cone-rod homeobox (CRX); Guanylate cyclase 2D (GUCY2D); Inosine monophosphate dehydrogenase 1 (IMPDH1); Lebercilin (LCAS); Lecithin retinol acyltransferase (LRAT); Retinal degeneration 3 (RD3); Retinol dehydrogenase 12 (RDH12); Retinal pigment epithelium specific protein 65 (RPE65); Retinitis pigmentosa GTPase regulator interacting protein 1 (RPGRIP1); Spermatogenesis associated 7 (SPATA7); or Tubby like protein 1 (TULP1).

Usually, the ZFPs include at least three fingers. Certain of the ZFPs include four, five or six fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, which domains can be transcriptional activation or repression domains.

In some embodiments, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-Scell, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 2007/0117128.

In other embodiments, the DNA binding domain comprises an engineered domain from a TAL effector similar to those derived from the plant pathogens Xanthomonas (see Boch et al, (2009) Science 326: 1509-1512 and Moscou and Bogdanove, (2009) Science 326: 1501) and Ralstonia (see Heuer et al (2007) Applied and Environmental Microbiology 73(13): 4379-4384); U.S. Patent Publication Nos. 2011/0301073 and 2011/0145940. The plant pathogenic bacteria of the genus Xanthomonas are known to cause many diseases in important crop plants. Pathogenicity of Xanthomonas depends on a conserved type III secretion (T3 S) system which injects more than 25 different effector proteins into the plant cell. Among these injected proteins are transcription activator-like effectors (TALE) which mimic plant transcriptional activators and manipulate the plant transcriptome (see Kay et al (2007) Science 318:648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet 218: 127-136 and International Publication No. WO 2010/079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TAL effectors depends on the sequences found in the tandem repeats. The repeated sequence comprises approximately 102 base pairs and the repeats are typically 91-100% homologous with each other (Bonas et al, ibid). Polymorphism of the repeats is usually located at positions 12 and 13 and there appears to be a one-to-one correspondence between the identity of the hypervariable diresidues (the repeat variable diresidue or RVD region) at positions 12 and 13 with the identity of the contiguous nucleotides in the TAL-effector's target sequence (see Moscou and Bogdanove, (2009) Science 326:1501 and Boch et al (2009) Science 326:1509-1512). Experimentally, the natural code for DNA recognition of these TAL-effectors has been determined such that an HD sequence at positions 12 and 13 (Repeat Variable Diresidue or RVD) leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN), including TALENs with atypical RVDs. See, e.g., U.S. Pat. No. 8,586,526.

In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224).

In still further embodiments, the nuclease comprises a compact TALEN. These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley et al (2013) Nat Comm: 1-8 DOI: 10.1038/ncomms2782). In addition, the nuclease domain may also exhibit DNA-binding functionality. Any TALENs may be used in combination with additional TALENs (e.g., one or more TALENs (cTALENs or FokI-TALENs) with one or more mega-TALEs.

In addition, as disclosed in these and other references, zinc finger domains and/or multi-fingered zinc finger proteins or TALEs may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in U.S. Pat. No. 6,794,136.

In certain embodiments, the DNA-binding domain is part of a CRISPR/Cas nuclease system, including a single guide RNA (sgRNA) that binds to DNA. See, e.g., U.S. Pat. No. 8,697,359 and U.S. Patent Publication Nos. 2015/0056705 and 2015/0159172. The CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1:e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs functional domain (e.g., nuclease such as Cas) to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof such as derivative Cas proteins. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein. In some embodiments, the Cas protein is a small Cas9 ortholog for delivery via an AAV vector (Ran et al (2015) Nature 510, p. 186).

In some embodiments, the DNA binding domain is part of a TtAgo system (see Swarts et al, ibid; Sheng et al, ibid). In eukaryotes, gene silencing is mediated by the Argonaute (Ago) family of proteins. In this paradigm, Ago is bound to small (19-31 nt) RNAs. This protein-RNA silencing complex recognizes target RNAs via Watson-Crick base pairing between the small RNA and the target and endonucleolytically cleaves the target RNA (Vogel (2014) Science 344:972-973). In contrast, prokaryotic Ago proteins bind to small single-stranded DNA fragments and likely function to detect and remove foreign (often viral) DNA (Yuan et al., (2005) Mol. Cell 19, 405; Olovnikov, et al. (2013) Mol. Cell 51, 594; Swarts et al., ibid). Exemplary prokaryotic Ago proteins include those from Aquifex aeolicus, Rhodobacter sphaeroides, and Thermus thermophilus.

One of the most well-characterized prokaryotic Ago protein is the one from T. thermophilus (TtAgo; Swarts et al. ibid). TtAgo associates with either 15 nt or 13-25 nt single-stranded DNA fragments with 5′ phosphate groups. This “guide DNA” bound by TtAgo serves to direct the protein-DNA complex to bind a Watson-Crick complementary DNA sequence in a third-party molecule of DNA. Once the sequence information in these guide DNAs has allowed identification of the target DNA, the TtAgo-guide DNA complex cleaves the target DNA. Such a mechanism is also supported by the structure of the TtAgo-guide DNA complex while bound to its target DNA (G. Sheng et al., ibid). Ago from Rhodobacter sphaeroides (RsAgo) has similar properties (Olivnikov et al. (2013) Mol. Cell. 51(5):594-605.

Exogenous guide DNAs of arbitrary DNA sequence can be loaded onto the TtAgo protein (Swarts et al. ibid.). Since the specificity of TtAgo cleavage is directed by the guide DNA, a TtAgo-DNA complex formed with an exogenous, investigator-specified guide DNA will therefore direct TtAgo target DNA cleavage to a complementary investigator-specified target DNA. In this way, one may create a targeted double-strand break in DNA. Use of the TtAgo-guide DNA system (or orthologous Ago-guide DNA systems from other organisms) allows for targeted cleavage of genomic DNA within cells. Such cleavage can be either single- or double-stranded. For cleavage of mammalian genomic DNA, it would be preferable to use of a version of TtAgo codon optimized for expression in mammalian cells. Further, it might be preferable to treat cells with a TtAgo-DNA complex formed in vitro where the TtAgo protein is fused to a cell-penetrating peptide. Further, it might be preferable to use a version of the TtAgo protein that has been altered via mutagenesis to have improved activity at 37° C. Ago-RNA-mediated DNA cleavage could be used to affect a panopoly of outcomes including gene knock-out, targeted gene addition, gene correction, targeted gene deletion using techniques standard in the art for exploitation of DNA breaks.

Thus, any DNA-binding domain can be used. The DNA-binding domains described herein typically bind to a target site comprising 12 to 35 nucleotides (or any value therebetween). The nucleotides within the target sites that are bound by the DNA-binding domain may be contiguous or non-contiguous (e.g., the DNA-binding domain may bind to less than all base pairs making up the target site).

Fusion Molecules

Fusion molecules comprising DNA-binding domains (e.g., ZFPs or TALEs, CRISPR/Cas components such as single guide RNAs) as described herein and a heterologous regulatory (functional) domain (or functional fragment thereof) are also provided. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, kinases, phosphatases, polymerases, endonucleases) and their associated factors and modifiers. U.S. Patent Publication Nos. 2005/0064474; 2006/0188987 and 2007/0218528 for details regarding fusions of DNA-binding domains and nuclease cleavage domains, incorporated by reference in their entireties herein.

Suitable domains for achieving activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Sp1, AP-2, and CTF1 (Seipel et al., EMBO J. 11, 4961-4968 (1992) as well as p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5, -6, -7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant 1 22:1-8; Gong et al. (1999) Plant Mol. Biol. 41:33-44; and Hobo et al. (1999) Proc. Natl. Acad. Sci. USA 96:15,348-15,353.

It will be clear to those of skill in the art that, in the formation of a fusion protein (or a nucleic acid encoding same) between a DNA-binding domain and a functional domain, either an activation domain or a molecule that interacts with an activation domain is suitable as a functional domain. Essentially any molecule capable of recruiting an activating complex and/or activating activity (such as, for example, histone acetylation) to the target gene is useful as an activating domain of a fusion protein. Insulator domains, localization domains, and chromatin remodeling proteins such as ISWI-containing domains and/or methyl binding domain proteins suitable for use as functional domains in fusion molecules are described, for example, in U.S. Patent Publication Nos. 2002/0115215 and 2003/0082552 and in International Publication No. WO 02/44376.

Exemplary repression domains include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al. (2000) Proc. Natl. Acad. Sci. USA 97:3930-3935. Furthermore, single guide RNAs of the CRISPR/Cas system associate with functional domains to form active transcriptional regulators and nucleases.

In certain embodiments, the target site is present in an accessible region of cellular chromatin. Accessible regions can be determined as described, for example, in U.S. Pat. Nos. 7,217,509 and 7,923,542. If the target site is not present in an accessible region of cellular chromatin, one or more accessible regions can be generated as described in U.S. Pat. Nos. 7,785,792 and 8,071,370. In additional embodiments, the DNA-binding domain of a fusion molecule is capable of binding to cellular chromatin regardless of whether its target site is in an accessible region or not. For example, such DNA-binding domains are capable of binding to linker DNA and/or nucleosomal DNA. Examples of this type of “pioneer” DNA binding domain are found in certain steroid receptor and in hepatocyte nuclear factor 3 (HNF3) (Cordingley et al. (1987) Cell 48:261-270; Pina et al. (1990) Cell 60:719-731; and Cirillo et al. (1998) EMBO J. 17:244-254).

The fusion molecule may be formulated with a pharmaceutically acceptable carrier, as is known to those of skill in the art. See, for example, Remington's Pharmaceutical Sciences, 17th ed., 1985; and U.S. Pat. Nos. 6,453,242 and 6,534,261.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers. In some embodiments, the functional domain enables the direct, irreversible conversion of one target DNA base into another in a programmable manner, without requiring dsDNA backbone cleavage or a donor template. In some aspects, the functional domain comprises cytidine deaminase activity, and mediates the direct conversion of a cytidine to a uridine, thereby effecting a C to T (or G to A) substitution. The resulting ‘base editors’ convert cytidines within a window of approximately five nucleotides of the site of DNA binding, and can efficiently cause a variety of point mutations relevant to human disease (see Komor et al, (2016) Nature April 20. doi: 10.1038/nature17946).

Additional exemplary functional domains are disclosed, for example, in U.S. Pat. Nos. 6,534,261 and 6,933,113.

Functional domains that are regulated by exogenous small molecules or ligands may also be selected. For example, RheoSwitch® technology may be employed wherein a functional domain only assumes its active conformation in the presence of the external RheoChem™ ligand (see for example U.S. Patent Publication No. 2009/0136465). Thus, the ZFP may be operably linked to the regulatable functional domain wherein the resultant activity of the ZFP-TF is controlled by the external ligand. Additional regulation can be accomplished through the use of transcriptional switches (e.g. small RNA or other types of controllable molecular switches (Aschrafi et al (2016) J Psychiatry Neurosci. 41(3): 150154)).

Nucleases

In certain embodiments, the fusion protein comprises a DNA-binding binding domain and cleavage (nuclease) domain. As such, gene modification can be achieved using a nuclease, for example an engineered nuclease. Engineered nuclease technology is based on the engineering of naturally occurring DNA-binding proteins. For example, engineering of homing endonucleases with tailored DNA-binding specificities has been described. Chames et al. (2005) Nucleic Acids Res 33(20):e178; Arnould et al. (2006) J. Mol. Biol. 355:443-458. In addition, engineering of ZFPs has also been described. See, e.g., U.S. Pat. Nos. 6,534,261; 6,607,882; 6,824,978; 6,979,539; 6,933,113; 7,163,824; and 7,013,219.

In addition, ZFPs and/or TALEs have been fused to nuclease domains to create ZFNs and TALENs—a functional entity that is able to recognize its intended nucleic acid target through its engineered (ZFP or TALE) DNA binding domain and cause the DNA to be cut near the DNA binding site via the nuclease activity. See, e.g., Kim et al. (1996) Proc Nat'l Acad Sci USA 93(3):1156-1160. More recently, such nucleases have been used for genome modification in a variety of organisms. See, for example, U.S. Patent Publication Nos. 2003/0232410; 2005/0208489; 2005/0026157; 2005/0064474; 2006/0188987; 2006/0063231; and International Publication No. WO 07/014275.

Thus, the methods and compositions described herein are broadly applicable and may involve any nuclease of interest. Non-limiting examples of nucleases include meganucleases, TALENs and zinc finger nucleases. The nuclease may comprise heterologous DNA-binding and cleavage domains (e.g., zinc finger nucleases; meganuclease DNA-binding domains with heterologous cleavage domains) or, alternatively, the DNA-binding domain of a naturally-occurring nuclease may be altered to bind to a selected target site (e.g., a meganuclease that has been engineered to bind to site different than the cognate binding site).

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site specific interaction with the target sequence of the user's choosing have been published (see U.S. Pat. No. 8,586,526). In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224). In addition, the nuclease domain may also exhibit DNA-binding functionality.

In still further embodiments, the nuclease comprises a compact TALEN (cTALEN). These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the TevI nuclease domain (see Beurdeley et al (2013) Nat Comm: 1-8 DOI: 10.1038/ncomms2782). Any TALENs may be used in combination with additional TALENs (e.g., one or more TALENs (cTALENs or FokI-TALENs) with one or more mega-TALs) or other DNA cleavage enzymes.

In certain embodiments, the nuclease comprises a meganuclease (homing endonuclease) or a portion thereof that exhibits cleavage activity. Naturally-occurring meganucleases recognize 15-40 base-pair cleavage sites and are commonly grouped into four families: the LAGLIDADG family, the GIY-YIG family, the His-Cys box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Duj on et al. (1989) Gene 82:115-118; Perler et al. (1994) Nucleic Acids Res. 22, 1125-1127; Jasin (1996) Trends Genet. 12:224-228; Gimble et al. (1996) J Mol. Biol. 263:163-180; Argast et al. (1998) J Mol. Biol. 280:345-353 and the New England Biolabs catalogue.

DNA-binding domains from naturally-occurring meganucleases, primarily from the LAGLIDADG family, have been used to promote site-specific genome modification in plants, yeast, Drosophila, mammalian cells and mice, but this approach has been limited to the modification of either homologous genes that conserve the meganuclease recognition sequence (Monet et al. (1999), Biochem. Biophysics. Res. Common. 255: 88-93) or to pre-engineered genomes into which a recognition sequence has been introduced (Route et al. (1994), Mol. Cell. Biol. 14: 8096-106; Chilton et al. (2003), Plant Physiology. 133: 956-65; Puchta et al. (1996), Proc. Natl. Acad. Sci. USA 93: 5055-60; Rong et al. (2002), Genes Dev. 16: 1568-81; Gouble et al. (2006), J. Gene Med. 8(5):616-622). Accordingly, attempts have been made to engineer meganucleases to exhibit novel binding specificity at medically or biotechnologically relevant sites (Porteus et al. (2005), Nat. Biotechnol. 23: 967-73; Sussman et al. (2004), J. Mol. Biol. 342: 31-41; Epinat et al. (2003), Nucleic Acids Res. 31: 2952-62; Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication Nos. 2007/0117128; 2006/0206949; 2006/0153826; 2006/0078552; and 2004/0002092). In addition, naturally-occurring or engineered DNA-binding domains from meganucleases can be operably linked with a cleavage domain from a heterologous nuclease (e.g., FokI) and/or cleavage domains from meganucleases can be operably linked with a heterologous DNA-binding domain (e.g., ZFP or TALE).

In other embodiments, the nuclease is a zinc finger nuclease (ZFN) or TALE DNA binding domain-nuclease fusion (TALEN). ZFNs and TALENs comprise a DNA binding domain (zinc finger protein or TALE DNA binding domain) that has been engineered to bind to a target site in a gene of choice and cleavage domain or a cleavage half-domain (e.g., from a restriction and/or meganuclease as described herein).

As described in detail above, zinc finger binding domains and TALE DNA binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain or TALE protein can have a novel binding specificity, compared to a naturally-occurring protein. Engineering methods include, but are not limited to, rational design and various types of selection. Rational design includes, for example, using databases comprising triplet (or quadruplet) nucleotide sequences and individual zinc finger or TALE amino acid sequences, in which each triplet or quadruplet nucleotide sequence is associated with one or more amino acid sequences of zinc fingers or TALE repeat units which bind the particular triplet or quadruplet sequence. See, for example, U.S. Pat. Nos. 6,453,242 and 6,534,261, incorporated by reference herein in their entireties.

Selection of target sites; and methods for design and construction of fusion proteins (and polynucleotides encoding same) are known to those of skill in the art and described in detail in U.S. Pat. Nos. 7,888,121 and 8,409,861, incorporated by reference in their entireties herein.

In addition, as disclosed in these and other references, zinc finger domains, TALEs and/or multi-fingered zinc finger proteins may be linked together using any suitable linker sequences, including for example, linkers of 5 or more amino acids in length. (e.g., TGEKP (SEQ ID NO:31), TGGQRP (SEQ ID NO:32), TGQKP (SEQ ID NO:33), and/or TGSQKP (SEQ ID NO:34)). See, e.g., U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. See, also, U.S. Pat. No. 8,772,453.

Thus, nucleases such as ZFNs, TALENs and/or meganucleases can comprise any DNA-binding domain and any nuclease (cleavage) domain (cleavage domain, cleavage half-domain). As noted above, the cleavage domain may be heterologous to the DNA-binding domain, for example a zinc finger or TAL-effector DNA-binding domain and a cleavage domain from a nuclease or a meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However, any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication No. WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Pat. Nos. 7,914,796; 8,034,598 and 8,623,618; and U.S. Patent Publication No. 2011/0201055, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Patent Publication No. 2008/0131962, the disclosure of which is incorporated by reference in its entirety for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. Nos. 7,914,796; 8,034,598 and 8,623,618, the disclosures of which are incorporated by reference in its entirety for all purposes. In other embodiments, the engineered cleavage half domain comprises the “Sharkey” and/or “Sharkey mutations” (see Guo et al, (2010) J. Mol. Biol. 400(1):96-107).

Alternatively, nucleases may be assembled in vivo at the nucleic acid target site using so-called “split-enzyme” technology (see e.g. U.S. Patent Publication No. 2009/0068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases (e.g., ZFNs and/or TALENs) can be screened for activity prior to use, for example in a yeast-based chromosomal system as described in as described in U.S. Pat. No. 8,563,314.

In certain embodiments, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In some embodiments, the CRISPR-Cpf1 system is used. The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al, (2015) Genom Bio 16:251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term “Cas” includes both Cas9 and Cfp1 proteins. Thus, as used herein, a “CRISPR/Cas system” refers both CRISPR/Cas and/or CRISPR/Cfp1 systems, including both nuclease and/or transcription factor systems.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

In some embodiments, the nuclease is a self-inactivating (see Epstein and Schaffer, (2016) ASGCT poster abstract 119).

The nuclease(s) may make one or more double-stranded and/or single-stranded cuts in the target site. In certain embodiments, the nuclease comprises a catalytically inactive cleavage domain (e.g., FokI and/or Cas protein). See, e.g., U.S. Pat. Nos. 9,200,266 and 8,703,489 and Guillinger et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffery et al. (2016) Nucleic Acids Res. 44(2):e11. doi: 10.1093/nar/gkv878. Epub 2015 Oct. 19.

Delivery

The proteins (e.g., nucleases and/or transcription factors), polynucleotides and/or compositions comprising the proteins and/or polynucleotides described herein may be delivered to a target cell by any suitable means, including, for example, by administration of the protein and/or polynucleotide (e.g., mRNA) components.

Suitable cells include but are not limited to eukaryotic and prokaryotic cells and/or cell lines. Non-limiting examples of such cells or cell lines generated from such cells include T-cells, COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NS0, SP2/0-Ag14, HeLa, HEK293 (e.g., HEK293-F, HEK293-H, HEK293-T), and perC6 cells as well as insect cells such as Spodoptera fugiperda (Sf), or fungal cells such as Saccharomyces, Pichia and Schizosaccharomyces. In certain embodiments, the cell line is a CHO-K1, MDCK or HEK293 cell line. Suitable cells also include stem cells such as, by way of example, embryonic stem cells, induced pluripotent stem cells (iPS cells), hematopoietic stem cells, neuronal stem cells, mesenchymal stem cells and bulge stem cells.

Methods of delivering proteins comprising DNA-binding domains as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

DNA binding domains and fusion proteins comprising these DNA binding domains as described herein may also be delivered using vectors containing sequences encoding one or more of the DNA-binding protein(s). Additionally, additional nucleic acids (e.g., donors) also may be delivered via these vectors. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more DNA-binding protein-encoding sequences and/or additional nucleic acids as appropriate. Thus, when one or more DNA-binding proteins as described herein are introduced into the cell, and additional DNAs as appropriate, they may be carried on the same vector or on different vectors. When multiple vectors are used, each vector may comprise a sequence encoding one or multiple DNA-binding proteins and additional nucleic acids as desired.

Conventional viral and non-viral based gene transfer methods can be used to introduce nucleic acids encoding engineered DNA-binding proteins in cells (e.g., mammalian cells) and target tissues and to co-introduce additional nucleotide sequences as desired. Such methods can also be used to administer nucleic acids (e.g., encoding DNA-binding proteins and/or donors) to cells in vitro. In certain embodiments, nucleic acids are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. In a preferred embodiment, one or more nucleic acids are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™, Lipofectin™, and Lipofectamine™ RNAiMAX). Cationic and neutral lipids that are suitable for efficient receptor-recognition lipofection of polynucleotides include those of Felgner, International Publication Nos. WO 91/17424 and WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al (2009) Nature Biotechnology 27(7) p. 643).

The use of RNA or DNA viral based systems for the delivery of nucleic acids encoding engineered DNA-binding proteins, and/or donors (e.g. CARs or ACTRs) as desired takes advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); International Publication No. WO 94/026877).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; International Publication NO. WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS USA 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS USA 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6, AAV8, AAV8.2, AAV9 and AAVrh10 and pseudotyped AAV such as AAV2/8, AAV2/5 and AAV2/6 can also be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In some embodiments, AAV is produced using a baculovirus expression system (see e.g. U.S. Pat. Nos. 6,723,551 and 7,271,002). Purification of AAV particles from a 293 or baculovirus system typically involves growth of the cells which produce the virus, followed by collection of the viral particles from the cell supernatant or lysing the cells and collecting the virus from the crude lysate. AAV is then purified by methods known in the art including ion exchange chromatography (e.g. see U.S. Pat. Nos. 7,419,817 and 6,989,264), ion exchange chromatography and CsCl density centrifugation (e.g. International Publication No. WO 2011/094198 A10), immunoaffinity chromatography (e.g. International Publication No. WO 2016/128408) or purification using AVB Sepharose (e.g. GE Healthcare Life Sciences).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., (Proc. Natl. Acad. Sci. USA 92:9747-9751 (1995)), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. In certain embodiments, the proteins and/or polynucleotides described herein are formulated in a pharmaceutical composition for delivery to the retina.

Alternatively, vectors can be delivered to cells ex vivo, such as cells explanted from an individual patient (e.g., lymphocytes, bone marrow aspirates, tissue biopsy) or universal donor hematopoietic stem cells, followed by re-implantation of the cells into a patient, usually after selection for cells which have incorporated the vector.

Ex vivo cell transfection for diagnostics, research, transplant or for gene therapy (e.g., via re-infusion of the transfected cells into the host organism) is well known to those of skill in the art. In a preferred embodiment, cells are isolated from the subject organism, transfected with a DNA-binding proteins nucleic acid (gene or cDNA), and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft, for example in the bone marrow or in the skin. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+(T cells), CD45+(panB cells), GR-1 (granulocytes), and Iad (differentiated antigen presenting cells) (see Inaba et al., J. Exp. Med. 176:1693-1702 (1992)).

Stem cells that have been modified may also be used in some embodiments. For example, stem cells that have been made resistant to apoptosis may be used as therapeutic compositions where the stem cells also contain modifications that induce resistance to apoptosis, for example, by knocking out BAX and/or BAK using BAX- or BAK-specific ZFNs (see, U.S. Pat. No. 8,597,912) in the stem cells, or those that are disrupted in a caspase, again using caspase-6 specific ZFNs for example.

Vectors (e.g., retroviruses, adenoviruses, liposomes, etc.) containing therapeutic DNA-binding proteins (or nucleic acids encoding these proteins) can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Heat may be used to increase delivery in conjunction with various administration methods. In preferred embodiments, administration directly to the site of treatment (e.g., retina) is performed. Suitable methods of administering (e.g., by subretinal injection) such nucleic acids, proteins and cells as described herein are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34+ cells, include adenovirus Type 35.

Vectors suitable for introduction of transgenes into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

In some embodiments, the therapeutic DNA-binding proteins can be delivered as polypeptides. In some instances, the therapeutic DNA-binding proteins can be delivered as polypeptides complexed to anionic nucleic acids. In some aspects, the proteins with or without bound nucleic acids are delivered using cationic lipid transfection reagents (Zuris et al (2015) Nat Biotechnol 33:73-80).

Pharmaceutically acceptable carriers are determined in part by the particular composition being administered, as well as by the particular method used to administer the composition. In certain embodiments, pharmaceutically acceptable carriers for topical administration are used. Accordingly, there is a wide variety of suitable formulations of pharmaceutical compositions available, as described below (see, e.g., Remington's Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells, including T-cells and stem cells of any type. Suitable cell lines for protein expression are known to those of skill in the art and include, but are not limited to COS, CHO (e.g., CHO-S, CHO-K1, CHO-DG44, CHO-DUXB11), VERO, MDCK, WI38, V79, B14AF28-G3, BHK, HaK, NS0, SP2/0-Ag14, HeLa, HEK293 (e.g., HEK293-F, HEK293-H, HEK293-T), perC6, insect cells such as Spodoptera fugiperda (Sf), and fungal cells such as Saccharomyces, Pichia and Schizosaccharomyces. Progeny, variants and derivatives of these cell lines can also be used.

Colloidal nanostructured lipid carriers (NLCs) represent a relatively new type of colloidal drug delivery system that consists of solid lipid and liquid lipid, and offers the advantage of improved drug loading capacity and release properties compared with solid lipid nanoparticles and via microneedles (Gomaa et al (2014) Eur. J Biopharm 86(2):145-155).

Subretinal injection can be accomplished by differing routes of administration. In some instances, subretinal injection is accomplished through a transscleral route where a need is advanced through the sclera, crossing through the vitreous, penetrating through the diametrically opposite retina into the subretinal space. Another route is the transscleral-transchoroidal-Bruch's membrane approach where subretinal injection is accomplished without penetrating the retina. Another possibility is injection into the subretinal space through a transcorneal route (Nickerson et al (2012) Methods Mol Bio 884:53-69).

Applications

The disclosed compositions and methods can be used for any application in which it is desired to modulate gene expression and/or functionality, including but not limited to, therapeutic and research applications in which gene modulation is desirable for the prevention or treatment of LCA. For example, the disclosed compositions can be used in vivo and/or ex vivo (cell therapies) to disrupt or repress the expression of a mutant CEP290 protein or to knock out a splicing signal in a mutant CEP290 coding sequence therapy thereby treating and/or preventing the LCA.

Methods and compositions also include stem cell compositions wherein the gene within the stem cells has been modulated (modified) and the cells further comprise an additional transgene. These altered stem cells are introduced into the patient through methods known in the art (e.g., through introduction into the subretinal space) to allow the engraftment of the cells in the patient. The introduced cells may also have other alterations to help during subsequent therapy.

The methods and compositions of the invention are also useful for the design and implementation of in vitro and in vivo models, for example, animal models of LCA and associated disorders, which allows for the study of these disorders.

All patents, patent applications and publications mentioned herein are hereby incorporated by reference in their entireties.

Although disclosure has been provided in some detail by way of illustration and example for the purposes of clarity and understanding, it will be apparent to those of skill in the art that various changes and modifications can be practiced without departing from the spirit or scope of the disclosure. Accordingly, the foregoing disclosure and following examples should not be construed as limiting.

### EXAMPLES

### Example 1: Design of Specific Nucleases

LCA related transcription factors and nucleases are constructed to enable modulation of one or more of the following genes: aryl hydrocarbon receptor interacting protein like 1 (AIPL1); Centrosomal protein 290 (CEP290); Crumbs1 cell polarity complex component (CRB1); Cone-rod homeobox (CRX); Guanylate cyclase 2D (GUCY2D); Inosine monophosphate dehydrogenase 1 (IMPDH1); Lebercilin (LCAS); Lecithin retinol acyltransferase (LRAT); Retinal degeneration 3 (RD3); Retinol dehydrogenase 12 (RDH12); Retinal pigment epithelium specific protein 65 (RPE65); Retinitis pigmentosa GTPase regulator interacting protein 1 (RPGRIP1); Spermatogenesis associated 7 (SPATA7); or Tubby like protein 1 (TULP1).

In particular, ZFNs and ZFP-TFs are designed essentially as described in Urnov et al. (2005) Nature 435(7042):646-651, Lombardo et al (2007) Nat Biotechnol. November; 25(11):1298-306, and U.S. Patent Publication Nos. 2008/0131962; 2015/0164954; 2014/0120622; 2014/0301990; and U.S. Pat. No. 8,956,828. All ZFNs are tested against their target gene in K562 cells and in human stem cells (skin stem cells) and found to be active.

Guide RNAs for the S. pyogenes CRISPR/Cas9 system are also constructed to target the genes listed above (see U.S. Patent Publication No. 2015/0056705), for example to a target site comprising 12 or more nucleotides of the target sites as shown in Table 1 All guide RNAs are tested in the CRISPR/Cas9 system and are found to be active in K562 cells.

TALENs and TALE-TFs are made to target the LCA-related gene(s) (see U.S. Pat. No. 8,586,526), for example TALENs or TALE-TFs that bind to a target site comprising 12 or more nucleotides of the target sites as shown in Table 1, and are tested in K562 cells and found to be active.

### Example 2: Activity of CEP290-Directed ZFN In Vitro

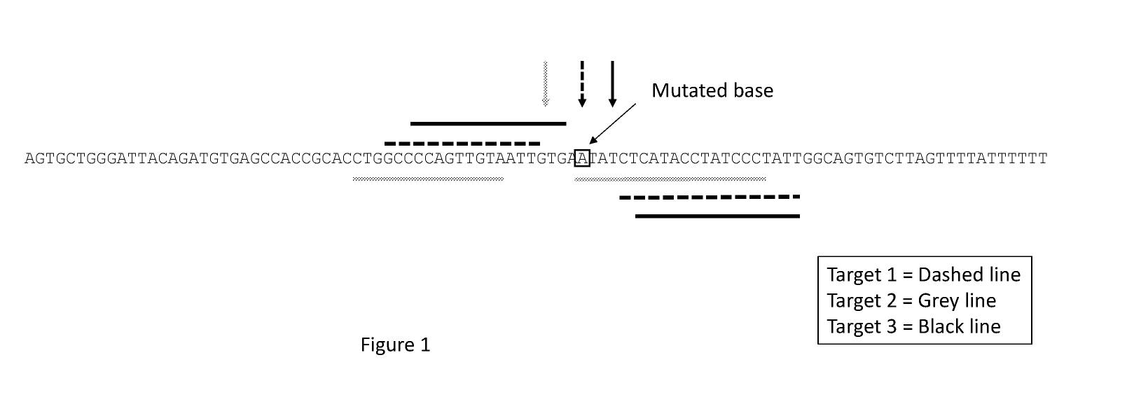

ZFNs were made to cleave the CEP290 gene within, at or near the site of c.2991+1655A>G mutation in intron 26. The ZFN pairs were designed to either cleave at the target base 1655 (Target #1), or to cleave 4 bp upstream (Target #2), or 3 bp downstream of the base (Target #3, See FIG. 1). Additional nucleases (e.g., TALENs and/or single RNAs of CRISPR/Cas systems) are designed to cleave at or near the mutation in intron 26, for example within, at or near a site comprising 12 or more (e.g., 12-35 or any value therebetween) contiguous nucleotides of the sequence shown in FIG. 1 (SEQ ID NO:35), including 12 or more contiguous nucleotides of the ZFN target sites shown below (e.g., 12 or more contiguous nucleotides of any of SEQ ID Nos:1-5).

The ZFNs and their target sites (bound nucleotides are shown in uppercase letters) are shown below in Table 1.

TABLE 1
ZFNs specific for CEP290

| | Design | | | | | | |
|---|---|---|---|---|---|---|---|
| | Human CEP290-specific ZFNs | | | | | | |
| SBS #,Target | F1 | F2 | F3 | F4 | F5 | F6 | Linker |
| SBS#61027 | TSSNRKT | QSAHRIT | RSDTLSQ | TSGHLSR | SLNSRYQ | RSAHLSR | N7a |
| 5′taGGGATAGGTA | (SEQ ID | (SEQ ID | (SEQ ID | (SEQ ID | (SEQ ID | (SEQ ID | |
| TGAGATATtcacaa | NO: 6) | NO: 7) | NO: 8) | NO: 9) | NO: 10) | NO: 11) | |
| tt (SEQ ID | | | | | | | |
| NO: 1) | | | | | | | |
| SBS#61024 | RSDALSV | DSSHRTR | RSDHLST | DRSNRKT | DRSNRIK | N/A | L0 |
| 5′ | (SEQ ID | (SEQ ID | (SEQ ID | (SEQ ID | (SEQ ID | | |
| atTACAACTGGGGC | NO: 12) | NO: 13) | NO: 14) | NO: 15) | NO: 16) | | |
| CAGgtgcggtggct | | | | | | | |
| (SEQ ID NO: 2) | | | | | | | |
| SBS#61033 | RSDNLAR | TSSNRKT | RSDNLSE | TSANLSR | RSDHLSQ | ASSNRIT | N7a |
| 5′ | (SEQ ID | (SEQ ID | (SEQ ID | (SEQ ID | (SEQ ID | (SEQ ID | |
| ccAATAGGGATAGG | NO: 17) | NO: 6) | NO: 18) | NO: 19) | NO: 20) | NO: 21) | |
| TATGAGatattcac | | | | | | | |
| (SEQ ID NO: 3) | | | | | | | |
| SBS#61028 | HSNARKT | QSGSLTR | TSGSLSR | QKGTLMS | DRSTRTK | N/A | N6a |
| 5′tgGCCCCAGTTG | (SEQ ID | (SEQ ID | (SEQ ID | (SEQ ID | (SEQ ID | | |
| TAATTgtgaatatc | NO: 22) | NO: 23) | NO: 24) | NO: 25) | NO: 26) | | |
| tc (SEQ ID | | | | | | | |
| NO: 4) | | | | | | | |
| SBS#61029 | HSNARKT | QSGSLTR | TSGSLSR | QKGTLMS | DRSTRTK | N/A | N7a |
| 5′tgGCCCCAGTTG | (SEQ ID | (SEQ ID | (SEQ ID | (SEQ ID | (SEQ ID | | |
| TAATTgtgaatatc | NO: 22) | NO: 23) | NO: 24) | NO: 25) | NO: 26) | | |
| tc (SEQ ID | | | | | | | |
| NO: 4) | | | | | | | |
| SBS#61035 | RSDNLAR | TSSNRKT | RSDNLSE | TSANLSR | RSDHLSQ | ASSNRIT | N6a |
| 5′ccAATAGGGATA | (SEQ ID | (SEQ ID | (SEQ ID | (SEQ ID | (SEQ ID | (SEQ ID | |
| GGTATGAGatattc | NO: 17) | NO: 6) | NO: 18) | NO: 19) | NO: 20) | NO: 21) | |
| ac(SEQ ID | | | | | | | |
| NO: 3) | | | | | | | |
| SBS#61041 | LKQNLDA | RSHHLKA | TSSNLSR | RSDHLSQ | ASSNRIT | N/A | N7a |
| 5′ccAATAGGGATA | (SEQ ID | (SEQ ID | (SEQ ID | (SEQ ID | (SEQ ID | | |
| GGTATgagatattc | NO: 27) | NO: 28) | NO: 29) | NO: 20) | NO: 21) | | |
| ac(SEQ ID | | | | | | | |
| NO: 3) | | | | | | | |
| SBS#61034 | RSASLLR | HSNARKT | QSGSLTR | TSGSLSR | QKGTLMS | N/A | N6a |
| 5′ccCCAGTTGTAA | (SEQ ID | (SEQ ID | (SEQ ID | (SEQ ID | (SEQ ID | | |
| TTGTGaatatctca | NO: 30) | NO: 22) | NO: 23) | NO: 24) | NO: 25) | | |
| ta(SEQ ID | | | | | | | |
| NO: 5) | | | | | | | |
| SBS#61036 | RSASLLR | HSNARKT | QSGSLTR | TSGSLSR | QKGTLMS | N/A | N7a |
| 5′ccCCAGTTGTAA | (SEQ ID | (SEQ ID | (SEQ ID | (SEQ ID | (SEQ ID | | |
| TTGTGaatatctca | NO: 30) | NO: 22) | NO: 23) | NO: 24) | NO: 25) | | |
| ta(SEQ ID | | | | | | | |
| NO: 5) | | | | | | | |
| SBS#61042 | LKQNLDA | RSHHLKA | TSSNLSR | RSDHLSQ | ASSNRIT | N/A | N6a |
| 5′ccAATAGGGATA | (SEQ ID | (SEQ ID | (SEQ ID | (SEQ ID | (SEQ ID | | |
| GGTATgagatattc | NO: 27) | NO: 28) | NO: 29) | NO: 20) | NO: 21) | | |
| ac(SEQ ID | | | | | | | |
| NO: 3) | | | | | | | |

The ZFNs were tested in pairs and shown to be active. Table 2 shows the cleavage activity of the proteins in K562 cells, assayed as described in Example 1. ZFNs were provided to the cells via electroporation of RNA. The amount of RNA used for each ZFN was 200 ng or 800 ng.

TABLE 2
Activity of CEP290-specific ZFN in vitro

| ZFN1 | ZFN2 | Target | % indels (200 ng) | % indels (800 ng) |
|---|---|---|---|---|
| 61027 | 61024 | 2 | 4.5 | 27.1 |
| 61033 | 61028 | 1 | 54.3 | 73.5 |
| 61033 | 61029 | 1 | 76.9 | 85.4 |
| 61035 | 61028 | 1 | 37.7 | 68.7 |
| 61035 | 61029 | 1 | 63.6 | 80.7 |
| 61041 | 61034 | 3 | 9.6 | 30.1 |
| 61041 | 61036 | 3 | 14.5 | 43.6 |
| 61042 | 61034 | 3 | 8.8 | 26.1 |
| 61042 | 61036 | 3 | 12.9 | 40.6 |
| control | control | control | 80.3 | 95.0 |

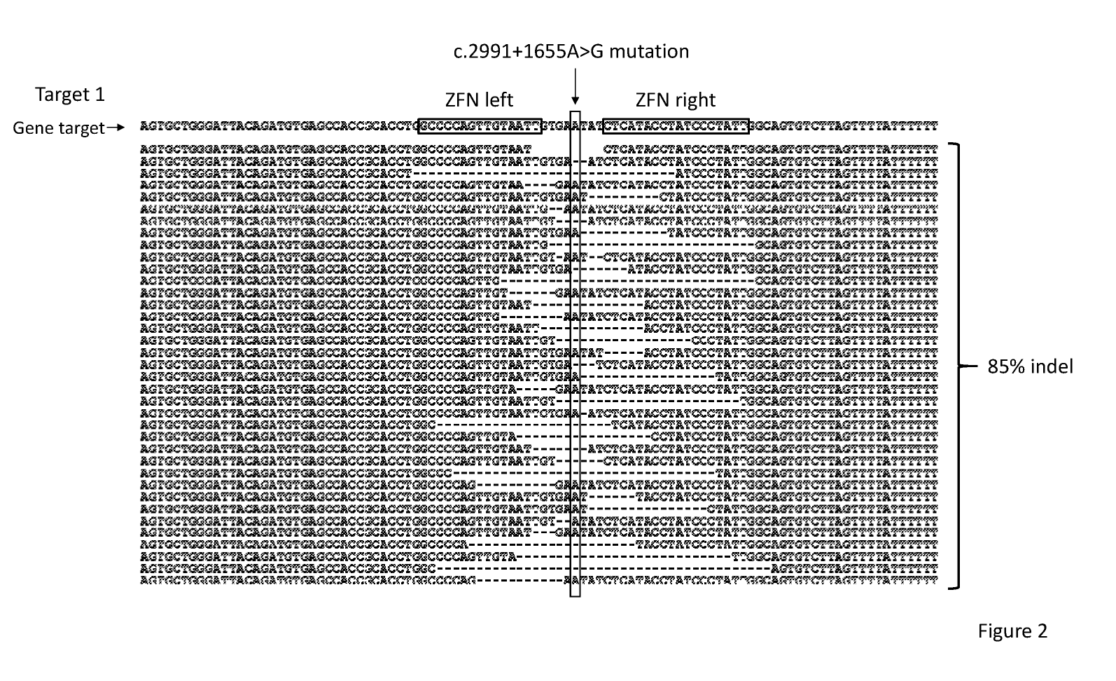

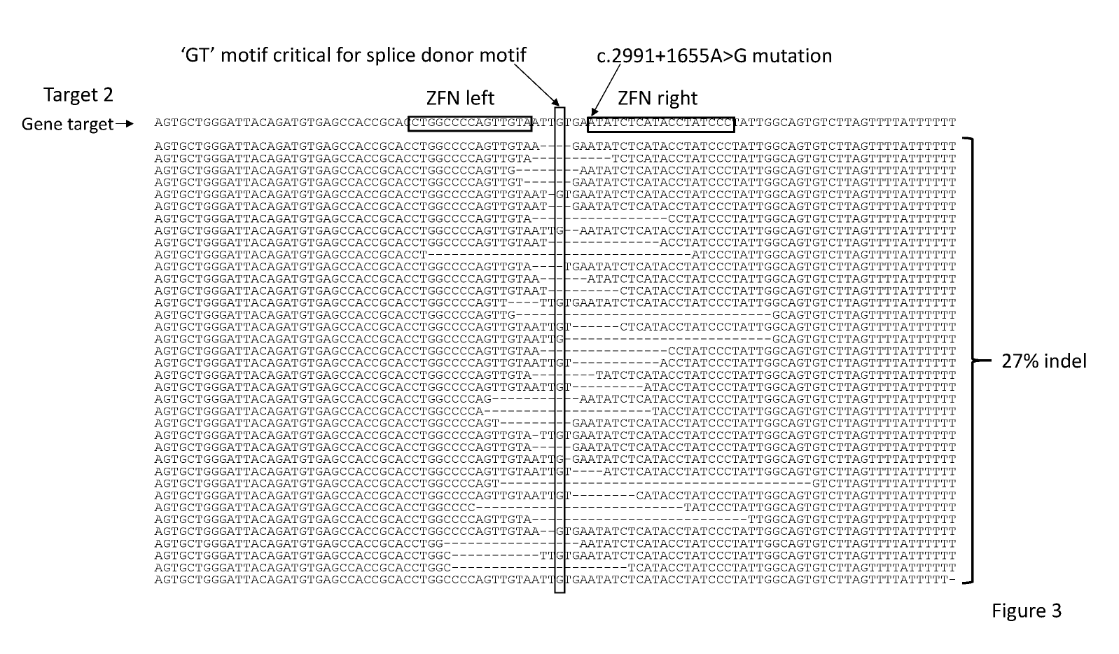

Amplicons encompassing the targeted region were amplified via PCR from the genomic DNA of treated K562 cells and submitted to MiSeq analysis. Resulting sequences were aligned to observe the location of indels at the cut site. The data demonstrate that the SBS#61033/SBS#61029 ZFN pair designed for Target 1 efficiently disrupts sequences at and in the vicinity of the base location of interest (FIG. 2, position 1655, boxed). The data also show that a ZFN pair designed for Target 2 (SBS#61027/SBS#61024) efficiently disrupts sequences at and in the vicinity of a nearby “GT” dinucleotide (FIG. 3, positions 1651-1652, boxed), which comprises a key conserved splice donor motif. It is likely that disruption of this dinucleotide would also abrogate aberrant splicing and restore normal expression to the CEP290 gene.

### Example 3: Activity of CEP290-Specific ZFN In Vivo

The highly active CEP290-specific ZFN are tested in vivo. A humanized mouse, where a segment of the human CEP290 gene comprising exon 26-intron 26 (with or without the c.2991+1655 A>G mutation)-exon 27 (Garanto et al (2013) PLoS One 8(11):e79369; Garanto et al (2016) Hum Mol Genet doi:10/1093/hmg/ddw118), has been inserted into the mouse CEP290 gene, is used to test the activity of the ZFN. Upon cleavage, the mutant A at position 1655 in the insert is altered following NHEJ of the cut DNA. The ZFNs are administered to the mouse retinas (e.g., via subretinal injection) either as mRNAs or are delivered packaged in AAV.

Analysis of the treated eyes reveals that CEP290 pre-mRNA splicing has been restored, and that CEP290 protein levels are increased as compared to the untreated mutant animals. In addition, ZFN treatment restores vision partially or fully in mouse models of LCA.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing description and examples should not be construed as limiting.

What is claimed is:

1. A modified cell in which expression of an aberrant endogenous Leber congenital amaurosis (LCA)-related gene is partially or completely restored as compared to a cell comprising an aberrant endogenous LCA-related gene that is not modified.

2. The modified cell of claim 1, wherein the LCA-related gene is an aryl hydrocarbon receptor interacting protein like 1 (AIPL1) gene; a Centrosomal protein 290 (CEP290) gene; a Crumbs1 cell polarity complex component (CRB1) gene; a Cone-rod homeobox (CRX) gene; a Guanylate cyclase 2D (GUCY2D) gene; an Inosine monophosphate dehydrogenase 1 (IMPDH1) gene; a Lebercilin (LCAS) gene; a Lecithin retinol acyltransferase (LRAT) gene; a Retinal degeneration 3 (RD3) gene; a Retinol dehydrogenase 12 (RDH12) gene; a Retinal pigment epithelium specific protein 65 (RPE65) gene; a Retinitis pigmentosa GTPase regulator interacting protein 1 (RPGRIP1) gene; a Spermatogenesis associated 7 (SPATA7) gene; or a Tubby like protein 1 (TULP1) gene.

3. The modified cell of claim 1, wherein the cell is genetically modified by insertion and/or deletion is induced by a nuclease.

4. The modified cell of claim 1, wherein the a CEP290 gene is genetically modified by introducing an insertion and/or deletion into the CEP290 gene.

5. The genetically modified cell of claim 4, wherein the insertion and/or deletion is within a target site as shown in Table 1 or within 300 nucleotides of a target site as shown in Table 1.

6. The genetically modified cell of claim 4, wherein the nuclease is a zinc finger nuclease, a TALEN or a CRISPR/Cas system.

7. The modified cell of claim 1, wherein the cell comprises an artificial transcription factor that alters expression of the LCA-related gene.

8. The modified cell of claim 7, wherein the artificial transcription factor comprises a transcriptional regulatory domain and a DNA-binding domain that binds to a target site comprising 12 or more base pairs as shown in Table 1.

9. The modified cell of claim 7, wherein the transcriptional regulatory domain comprises a repression domain.

10. A pharmaceutical composition comprising a modified cell of claim 1.

11. A method of treating or preventing LCA in a subject, the method comprising:
administering a nuclease that modifies an LCA-related gene to generate a genetically modified cell according to claim 3, wherein the cell is in the subject and LCA is treated or prevented.

12. The method of claim 11, wherein the genetically modified cell is generated in vitro and administered to the subject.

13. The method of claim 12, wherein cell is administered via topical application or via injection into the area to be treated.

14. A composition for modifying one or more aberrant endogenous LCA-related genes in a cell of a mammalian subject, the composition comprising one or more nucleases, the one or more nuclease(s) comprising a DNA-binding domain that binds to the aberrant endogenous LCA-related gene and an endonuclease cleavage domain, wherein the nuclease cleaves the one or more endogenous LCA-related gene.

15. The composition of claim 14, wherein the modification is selected from the group consisting of an insertion, a deletion, a substitution and combinations thereof.

16. The composition of claim 14, wherein the expression of the one or more aberrant endogenous LCA-related genes is partially or completely restored.

17. A composition according to claim 14, characterized in that the composition modifies an aberrant endogenous LCA-related gene in one or more cells of the subject.

18. The composition of claim 17, wherein the LCA-related gene is CEP290.

19. The composition according to claim 14, wherein the subject is suffering from LCA.

20. A method of treating LCA in a subject comprising administering a composition according to claim 14 to the subject.

21. The method of claim 17, wherein the composition is administered to the eye.

22. A method of treating LCA in a subject, the method comprising generating a cell according to claim 7 in the subject by administering to the subject an artificial transcription factor that alters expression of an LCA-related gene.


SEQUENCE LISTING (113 sequences)

SEQ ID NO: 1 - DNA, 28 bp, Artificial Sequence
  Description of Artificial Sequence Synthetic oligonucleotide
tagggataggtatgagatattcacaatt

SEQ ID NO: 2 - DNA, 28 bp, Artificial Sequence
  Description of Artificial Sequence Synthetic oligonucleotide
attacaactggggccaggtgcggtggct

SEQ ID NO: 3 - DNA, 28 bp, Artificial Sequence
  Description of Artificial Sequence Synthetic oligonucleotide
ccaatagggataggtatgagatattcac

SEQ ID NO: 4 - DNA, 28 bp, Artificial Sequence
  Description of Artificial Sequence Synthetic oligonucleotide
tggccccagttgtaattgtgaatatctc

SEQ ID NO: 5 - DNA, 28 bp, Artificial Sequence
  Description of Artificial Sequence Synthetic oligonucleotide
ccccagttgtaattgtgaatatctcata

SEQ ID NO: 6 - protein, 7 aa, Artificial Sequence
  Description of Artificial Sequence Synthetic peptide
TSSNRKT

SEQ ID NO: 7 - protein, 7 aa, Artificial Sequence
  Description of Artificial Sequence Synthetic peptide
QSAHRIT

SEQ ID NO: 8 - protein, 7 aa, Artificial Sequence
  Description of Artificial Sequence Synthetic peptide
RSDTLSQ

SEQ ID NO: 9 - protein, 7 aa, Artificial Sequence
  Description of Artificial Sequence Synthetic peptide
TSGHLSR

SEQ ID NO: 10 - protein, 7 aa, Artificial Sequence
  Description of Artificial Sequence Synthetic peptide
SLNSRYQ

SEQ ID NO: 11 - protein, 7 aa, Artificial Sequence
  Description of Artificial Sequence Synthetic peptide
RSAHLSR

SEQ ID NO: 12 - protein, 7 aa, Artificial Sequence
  Description of Artificial Sequence Synthetic peptide
RSDALSV

SEQ ID NO: 13 - protein, 7 aa, Artificial Sequence
  Description of Artificial Sequence Synthetic peptide
DSSHRTR

SEQ ID NO: 14 - protein, 7 aa, Artificial Sequence
  Description of Artificial Sequence Synthetic peptide
RSDHLST

SEQ ID NO: 15 - protein, 7 aa, Artificial Sequence
  Description of Artificial Sequence Synthetic peptide
DRSNRKT

SEQ ID NO: 16 - protein, 7 aa, Artificial Sequence
  Description of Artificial Sequence Synthetic peptide
DRSNRIK

SEQ ID NO: 17 - protein, 7 aa, Artificial Sequence
  Description of Artificial Sequence Synthetic peptide
RSDNLAR

SEQ ID NO: 18 - protein, 7 aa, Artificial Sequence
  Description of Artificial Sequence Synthetic peptide
RSDNLSE

SEQ ID NO: 19 - protein, 7 aa, Artificial Sequence
  Description of Artificial Sequence Synthetic peptide
TSANLSR

SEQ ID NO: 20 - protein, 7 aa, Artificial Sequence
  Description of Artificial Sequence Synthetic peptide
RSDHLSQ

SEQ ID NO: 21 - protein, 7 aa, Artificial Sequence
  Description of Artificial Sequence Synthetic peptide
ASSNRIT

SEQ ID NO: 22 - protein, 7 aa, Artificial Sequence
  Description of Artificial Sequence Synthetic peptide
HSNARKT

SEQ ID NO: 23 - protein, 7 aa, Artificial Sequence
  Description of Artificial Sequence Synthetic peptide
QSGSLTR

SEQ ID NO: 24 - protein, 7 aa, Artificial Sequence
  Description of Artificial Sequence Synthetic peptide
TSGSLSR

SEQ ID NO: 25 - protein, 7 aa, Artificial Sequence
  Description of Artificial Sequence Synthetic peptide
QKGTLMS

SEQ ID NO: 26 - protein, 7 aa, Artificial Sequence
  Description of Artificial Sequence Synthetic peptide
DRSTRTK

SEQ ID NO: 27 - protein, 7 aa, Artificial Sequence
  Description of Artificial Sequence Synthetic peptide
LKQNLDA

SEQ ID NO: 28 - protein, 7 aa, Artificial Sequence
  Description of Artificial Sequence Synthetic peptide
RSHHLKA

SEQ ID NO: 29 - protein, 7 aa, Artificial Sequence
  Description of Artificial Sequence Synthetic peptide
TSSNLSR

SEQ ID NO: 30 - protein, 7 aa, Artificial Sequence
  Description of Artificial Sequence Synthetic peptide
RSASLLR

SEQ ID NO: 31 - protein, 5 aa, Artificial Sequence
  Description of Artificial Sequence Synthetic peptide
TGEKP

SEQ ID NO: 32 - protein, 6 aa, Artificial Sequence
  Description of Artificial Sequence Synthetic peptide
TGGQRP

SEQ ID NO: 33 - protein, 5 aa, Artificial Sequence
  Description of Artificial Sequence Synthetic peptide
TGQKP

SEQ ID NO: 34 - protein, 6 aa, Artificial Sequence
  Description of Artificial Sequence Synthetic peptide
TGSQKP

SEQ ID NO: 35 - DNA, 100 bp, Artificial Sequence
  Description of Artificial Sequence Synthetic polynucleotide
agtgctgggattacagatgtgagccaccgcacctggccccagttgtaattgtgaatatctcatacctatccctattggcagtgtcttagttttatttttt

SEQ ID NO: 36 - DNA, 100 bp, Artificial Sequence; identical to SEQ ID NO: 35
  Description of Artificial Sequence Synthetic polynucleotide

SEQ ID NO: 37 - DNA, 49 bp, Artificial Sequence
  Description of Artificial Sequence Synthetic oligonucleotide
agtgctgggattacagatgtgagccaccgcacctggccccagttgtaat

SEQ ID NO: 38 - DNA, 98 bp, Artificial Sequence
  Description of Artificial Sequence Synthetic oligonucleotide
agtgctgggattacagatgtgagccaccgcacctggccccagttgtaattgtgaatctcatacctatccctattggcagtgtcttagttttatttttt

SEQ ID NO: 39 - DNA, 67 bp, Artificial Sequence
  Description of Artificial Sequence Synthetic oligonucleotide
agtgctgggattacagatgtgagccaccgcacctatccctattggcagtgtcttagttttatttttt

SEQ ID NO: 40 - DNA, 96 bp, Artificial Sequence
  Description of Artificial Sequence Synthetic oligonucleotide
agtgctgggattacagatgtgagccaccgcacctggccccagttgtaagaatatctcatacctatccctattggcagtgtcttagttttatttttt

SEQ ID NO: 41 - DNA, 91 bp, Artificial Sequence
  Description of Artificial Sequence Synthetic oligonucleotide
agtgctgggattacagatgtgagccaccgcacctggccccagttgtaattgtgaatctatccctattggcagtgtcttagttttatttttt

SEQ ID NO: 42 - DNA, 98 bp, Artificial Sequence
  Description of Artificial Sequence Synthetic oligonucleotide
agtgctgggattacagatgtgagccaccgcacctggccccagttgtaattgaatatctcatacctatccctattggcagtgtcttagttttatttttt

SEQ ID NO: 43 - DNA, 96 bp, Artificial Sequence
  Description of Artificial Sequence Synthetic oligonucleotide
agtgctgggattacagatgtgagccaccgcacctggccccagttgtaattgtatctcatacctatccctattggcagtgtcttagttttatttttt

SEQ ID NO: 44 - DNA, 89 bp, Artificial Sequence
  Description of Artificial Sequence Synthetic oligonucleotide
agtgctgggattacagatgtgagccaccgcacctggccccagttgtaattgtgaatatccctattggcagtgtcttagttttatttttt

SEQ ID NO: 45 - DNA, 74 bp, Artificial Sequence
  Description of Artificial Sequence Synthetic oligonucleotide
agtgctgggattacagatgtgagccaccgcacctggccccagttgtaattggcagtgtcttagttttatttttt

SEQ ID NO: 46 - DNA, 97 bp, Artificial Sequence
  Description of Artificial Sequence Synthetic oligonucleotide
agtgctgggattacagatgtgagccaccgcacctggccccagttgtaattgtaatctcatacctatccctattggcagtgtcttagttttatttttt

SEQ ID NO: 47 - DNA, 93 bp, Artificial Sequence
  Description of Artificial Sequence Synthetic oligonucleotide
agtgctgggattacagatgtgagccaccgcacctggccccagttgtaattgtgaatacctatccctattggcagtgtcttagttttatttttt

SEQ ID NO: 48 - DNA, 68 bp, Artificial Sequence
  Description of Artificial Sequence Synthetic oligonucleotide
agtgctgggattacagatgtgagccaccgcacctggccccagttggcagtgtcttagttttatttttt

SEQ ID NO: 49 - DNA, 94 bp, Artificial Sequence
  Description of Artificial Sequence Synthetic oligonucleotide
agtgctgggattacagatgtgagccaccgcacctggccccagttgtgaatatctcatacctatccctattggcagtgtcttagttttatttttt

SEQ ID NO: 50 - DNA, 86 bp, Artificial Sequence
  Description of Artificial Sequence Synthetic oligonucleotide
agtgctgggattacagatgtgagccaccgcacctggccccagttgtaatacctatccctattggcagtgtcttagttttatttttt

SEQ ID NO: 51 - DNA, 92 bp, Artificial Sequence
  Description of Artificial Sequence Synthetic oligonucleotide
agtgctgggattacagatgtgagccaccgcacctggccccagttgaatatctcatacctatccctattggcagtgtcttagttttatttttt

SEQ ID NO: 52 - DNA, 87 bp, Artificial Sequence
  Description of Artificial Sequence Synthetic oligonucleotide
agtgctgggattacagatgtgagccaccgcacctggccccagttgtaattacctatccctattggcagtgtcttagttttatttttt

SEQ ID NO: 53 - DNA, 83 bp, Artificial Sequence
  Description of Artificial Sequence Synthetic oligonucleotide
agtgctgggattacagatgtgagccaccgcacctggccccagttgtaattgtccctattggcagtgtcttagttttatttttt

SEQ ID NO: 54 - DNA, 95 bp, Artificial Sequence
  Description of Artificial Sequence Synthetic oligonucleotide
agtgctgggattacagatgtgagccaccgcacctggccccagttgtaattgtgaatatacctatccctattggcagtgtcttagttttatttttt

SEQ ID NO: 55 - DNA, 97 bp, Artificial Sequence
  Description of Artificial Sequence Synthetic oligonucleotide
agtgctgggattacagatgtgagccaccgcacctggccccagttgtaattgtgatctcatacctatccctattggcagtgtcttagttttatttttt

SEQ ID NO: 56 - DNA, 83 bp, Artificial Sequence
  Description of Artificial Sequence Synthetic oligonucleotide
agtgctgggattacagatgtgagccaccgcacctggccccagttgtaattgtgaatattggcagtgtcttagttttatttttt

SEQ ID NO: 57 - DNA, 95 bp, Artificial Sequence
  Description of Artificial Sequence Synthetic oligonucleotide
agtgctgggattacagatgtgagccaccgcacctggccccagttgtagaatatctcatacctatccctattggcagtgtcttagttttatttttt

SEQ ID NO: 58 - DNA, 77 bp, Artificial Sequence
  Description of Artificial Sequence Synthetic oligonucleotide
agtgctgggattacagatgtgagccaccgcacctggccccagttgtaattgttggcagtgtcttagttttatttttt

SEQ ID NO: 59 - DNA, 99 bp, Artificial Sequence
  Description of Artificial Sequence Synthetic oligonucleotide
agtgctgggattacagatgtgagccaccgcacctggccccagttgtaattgtgaaatctcatacctatccctattggcagtgtcttagttttatttttt

SEQ ID NO: 60 - DNA, 78 bp, Artificial Sequence
  Description of Artificial Sequence Synthetic oligonucleotide
agtgctgggattacagatgtgagccaccgcacctggctcatacctatccctattggcagtgtcttagttttatttttt

SEQ ID NO: 61 - DNA, 83 bp, Artificial Sequence
  Description of Artificial Sequence Synthetic oligonucleotide
agtgctgggattacagatgtgagccaccgcacctggccccagttgtacctatccctattggcagtgtcttagttttatttttt

SEQ ID NO: 62 - DNA, 93 bp, Artificial Sequence
  Description of Artificial Sequence Synthetic oligonucleotide
agtgctgggattacagatgtgagccaccgcacctggccccagttgtaatatctcatacctatccctattggcagtgtcttagttttatttttt

SEQ ID NO: 63 - DNA, 94 bp, Artificial Sequence
  Description of Artificial Sequence Synthetic oligonucleotide
agtgctgggattacagatgtgagccaccgcacctggccccagttgtaattgtctcatacctatccctattggcagtgtcttagttttatttttt

SEQ ID NO: 64 - DNA, 67 bp, Artificial Sequence
  Description of Artificial Sequence Synthetic oligonucleotide
agtgctgggattacagatgtgagccaccgcacctggccctattggcagtgtcttagttttatttttt

SEQ ID NO: 65 - DNA, 90 bp, Artificial Sequence
  Description of Artificial Sequence Synthetic oligonucleotide
agtgctgggattacagatgtgagccaccgcacctggccccaggaatatctcatacctatccctattggcagtgtcttagttttatttttt

SEQ ID NO: 66 - DNA, 94 bp, Artificial Sequence
  Description of Artificial Sequence Synthetic oligonucleotide
agtgctgggattacagatgtgagccaccgcacctggccccagttgtaattgtgaattacctatccctattggcagtgtcttagttttatttttt

SEQ ID NO: 67 - DNA, 85 bp, Artificial Sequence
  Description of Artificial Sequence Synthetic oligonucleotide
agtgctgggattacagatgtgagccaccgcacctggccccagttgtaattgtgaatctattggcagtgtcttagttttatttttt

SEQ ID NO: 68 - DNA, 98 bp, Artificial Sequence
  Description of Artificial Sequence Synthetic oligonucleotide
agtgctgggattacagatgtgagccaccgcacctggccccagttgtaattgtatatctcatacctatccctattggcagtgtcttagttttatttttt

SEQ ID NO: 69 - DNA, 97 bp, Artificial Sequence
  Description of Artificial Sequence Synthetic oligonucleotide
agtgctgggattacagatgtgagccaccgcacctggccccagttgtaatgaatatctcatacctatccctattggcagtgtcttagttttatttttt

SEQ ID NO: 70 - DNA, 79 bp, Artificial Sequence
  Description of Artificial Sequence Synthetic oligonucleotide
agtgctgggattacagatgtgagccaccgcacctggccccatacctatccctattggcagtgtcttagttttatttttt

SEQ ID NO: 71 - DNA, 73 bp, Artificial Sequence
  Description of Artificial Sequence Synthetic oligonucleotide
agtgctgggattacagatgtgagccaccgcacctggccccagttgtattggcagtgtcttagttttatttttt

SEQ ID NO: 72 - DNA, 58 bp, Artificial Sequence
  Description of Artificial Sequence Synthetic oligonucleotide
agtgctgggattacagatgtgagccaccgcacctggcagtgtcttagttttatttttt

SEQ ID NO: 73 - DNA, 89 bp, Artificial Sequence
  Description of Artificial Sequence Synthetic oligonucleotide
agtgctgggattacagatgtgagccaccgcacctggccccagaatatctcatacctatccctattggcagtgtcttagttttatttttt

SEQ ID NO: 74 - DNA, 100 bp, Artificial Sequence; identical to SEQ ID NO: 35
  Description of Artificial Sequence Synthetic polynucleotide

SEQ ID NO: 75 - DNA, 96 bp, Artificial Sequence; identical to SEQ ID NO: 40
  Description of Artificial Sequence Synthetic oligonucleotide

SEQ ID NO: 76 - DNA, 90 bp, Artificial Sequence
  Description of Artificial Sequence Synthetic oligonucleotide
agtgctgggattacagatgtgagccaccgcacctggccccagttgtatctcatacctatccctattggcagtgtcttagttttatttttt

SEQ ID NO: 77 - DNA, 92 bp, Artificial Sequence; identical to SEQ ID NO: 51
  Description of Artificial Sequence Synthetic oligonucleotide

SEQ ID NO: 78 - DNA, 94 bp, Artificial Sequence; identical to SEQ ID NO: 49
  Description of Artificial Sequence Synthetic oligonucleotide

SEQ ID NO: 79 - DNA, 99 bp, Artificial Sequence
  Description of Artificial Sequence Synthetic oligonucleotide
agtgctgggattacagatgtgagccaccgcacctggccccagttgtaatgtgaatatctcatacctatccctattggcagtgtcttagttttatttttt

SEQ ID NO: 80 - DNA, 97 bp, Artificial Sequence; identical to SEQ ID NO: 69
  Description of Artificial Sequence Synthetic oligonucleotide

SEQ ID NO: 81 - DNA, 83 bp, Artificial Sequence; identical to SEQ ID NO: 61
  Description of Artificial Sequence Synthetic oligonucleotide

SEQ ID NO: 82 - DNA, 98 bp, Artificial Sequence; identical to SEQ ID NO: 42
  Description of Artificial Sequence Synthetic oligonucleotide

SEQ ID NO: 83 - DNA, 86 bp, Artificial Sequence; identical to SEQ ID NO: 50
  Description of Artificial Sequence Synthetic oligonucleotide

SEQ ID NO: 84 - DNA, 67 bp, Artificial Sequence; identical to SEQ ID NO: 39
  Description of Artificial Sequence Synthetic oligonucleotide

SEQ ID NO: 85 - DNA, 96 bp, Artificial Sequence
  Description of Artificial Sequence Synthetic oligonucleotide
agtgctgggattacagatgtgagccaccgcacctggccccagttgtatgaatatctcatacctatccctattggcagtgtcttagttttatttttt

SEQ ID NO: 86 - DNA, 94 bp, Artificial Sequence
  Description of Artificial Sequence Synthetic oligonucleotide
agtgctgggattacagatgtgagccaccgcacctggccccagttgtaaatatctcatacctatccctattggcagtgtcttagttttatttttt

SEQ ID NO: 87 - DNA, 91 bp, Artificial Sequence
  Description of Artificial Sequence Synthetic oligonucleotide
agtgctgggattacagatgtgagccaccgcacctggccccagttgtaatctcatacctatccctattggcagtgtcttagttttatttttt

SEQ ID NO: 88 - DNA, 96 bp, Artificial Sequence
  Description of Artificial Sequence Synthetic oligonucleotide
agtgctgggattacagatgtgagccaccgcacctggccccagttttgtgaatatctcatacctatccctattggcagtgtcttagttttatttttt

SEQ ID NO: 89 - DNA, 68 bp, Artificial Sequence; identical to SEQ ID NO: 48
  Description of Artificial Sequence Synthetic oligonucleotide

SEQ ID NO: 90 - DNA, 94 bp, Artificial Sequence; identical to SEQ ID NO: 63
  Description of Artificial Sequence Synthetic oligonucleotide

SEQ ID NO: 91 - DNA, 74 bp, Artificial Sequence; identical to SEQ ID NO: 45
  Description of Artificial Sequence Synthetic oligonucleotide

SEQ ID NO: 92 - DNA, 84 bp, Artificial Sequence
  Description of Artificial Sequence Synthetic oligonucleotide
agtgctgggattacagatgtgagccaccgcacctggccccagttgtaacctatccctattggcagtgtcttagttttatttttt

SEQ ID NO: 93 - DNA, 89 bp, Artificial Sequence
  Description of Artificial Sequence Synthetic oligonucleotide
agtgctgggattacagatgtgagccaccgcacctggccccagttgtaattgtacctatccctattggcagtgtcttagttttatttttt

SEQ ID NO: 94 - DNA, 92 bp, Artificial Sequence
  Description of Artificial Sequence Synthetic oligonucleotide
agtgctgggattacagatgtgagccaccgcacctggccccagttgtatatctcatacctatccctattggcagtgtcttagttttatttttt

SEQ ID NO: 95 - DNA, 91 bp, Artificial Sequence
  Description of Artificial Sequence Synthetic oligonucleotide
agtgctgggattacagatgtgagccaccgcacctggccccagttgtaattgtatacctatccctattggcagtgtcttagttttatttttt

SEQ ID NO: 96 - DNA, 89 bp, Artificial Sequence; identical to SEQ ID NO: 73
  Description of Artificial Sequence Synthetic oligonucleotide

SEQ ID NO: 97 - DNA, 79 bp, Artificial Sequence; identical to SEQ ID NO: 70
  Description of Artificial Sequence Synthetic oligonucleotide

SEQ ID NO: 98 - DNA, 91 bp, Artificial Sequence
  Description of Artificial Sequence Synthetic oligonucleotide
agtgctgggattacagatgtgagccaccgcacctggccccagtgaatatctcatacctatccctattggcagtgtcttagttttatttttt

SEQ ID NO: 99 - DNA, 99 bp, Artificial Sequence
  Description of Artificial Sequence Synthetic oligonucleotide
agtgctgggattacagatgtgagccaccgcacctggccccagttgtattgtgaatatctcatacctatccctattggcagtgtcttagttttatttttt

SEQ ID NO: 100 - DNA, 95 bp, Artificial Sequence; identical to SEQ ID NO: 57
  Description of Artificial Sequence Synthetic oligonucleotide

SEQ ID NO: 101 - DNA, 99 bp, Artificial Sequence
  Description of Artificial Sequence Synthetic oligonucleotide
agtgctgggattacagatgtgagccaccgcacctggccccagttgtaattggaatatctcatacctatccctattggcagtgtcttagttttatttttt

SEQ ID NO: 102 - DNA, 96 bp, Artificial Sequence; identical to SEQ ID NO: 43
  Description of Artificial Sequence Synthetic oligonucleotide

SEQ ID NO: 103 - DNA, 61 bp, Artificial Sequence
  Description of Artificial Sequence Synthetic oligonucleotide
agtgctgggattacagatgtgagccaccgcacctggccccagtgtcttagttttatttttt

SEQ ID NO: 104 - DNA, 92 bp, Artificial Sequence
  Description of Artificial Sequence Synthetic oligonucleotide
agtgctgggattacagatgtgagccaccgcacctggccccagttgtaattgtcatacctatccctattggcagtgtcttagttttatttttt

SEQ ID NO: 105 - DNA, 74 bp, Artificial Sequence
  Description of Artificial Sequence Synthetic oligonucleotide
agtgctgggattacagatgtgagccaccgcacctggcccctatccctattggcagtgtcttagttttatttttt

SEQ ID NO: 106 - DNA, 73 bp, Artificial Sequence; identical to SEQ ID NO: 71
  Description of Artificial Sequence Synthetic oligonucleotide

SEQ ID NO: 107 - DNA, 98 bp, Artificial Sequence
  Description of Artificial Sequence Synthetic oligonucleotide
agtgctgggattacagatgtgagccaccgcacctggccccagttgtaagtgaatatctcatacctatccctattggcagtgtcttagttttatttttt

SEQ ID NO: 108 - DNA, 83 bp, Artificial Sequence
  Description of Artificial Sequence Synthetic oligonucleotide
agtgctgggattacagatgtgagccaccgcacctggaatatctcatacctatccctattggcagtgtcttagttttatttttt

SEQ ID NO: 109 - DNA, 89 bp, Artificial Sequence
  Description of Artificial Sequence Synthetic oligonucleotide
agtgctgggattacagatgtgagccaccgcacctggcttgtgaatatctcatacctatccctattggcagtgtcttagttttatttttt

SEQ ID NO: 110 - DNA, 78 bp, Artificial Sequence; identical to SEQ ID NO: 60
  Description of Artificial Sequence Synthetic oligonucleotide

SEQ ID NO: 111 - DNA, 99 bp, Artificial Sequence
  Description of Artificial Sequence Synthetic oligonucleotide
agtgctgggattacagatgtgagccaccgcacctggccccagttgtaattgtgaatatctcatacctatccctattggcagtgtcttagttttattttt

SEQ ID NO: 112 - protein, 9 aa, Unknown
  Description of Unknown ′LAGLIDADG′ family motif peptide
LAGLIDADG

SEQ ID NO: 113 - DNA, 42 bp, Artificial Sequence
  Description of Artificial Sequence Synthetic oligonucleotide
ctcatacctatccctattggcagtgtcttagttttatttttt